(19) United States
(12) Patent Application Publication
(10) Pub. No.: US 2025/0083136 A1
(43) Pub. Date: Mar. 13, 2025

(54) METHODS AND SYSTEMS FOR MODIFYING SELECTIVITY IN CATALYTIC REACTIONS

(71) Applicant: Duke University, Durham, NC (US)

(72) Inventors: Ross A. Widenhoefer, Durham, NC (US); Xujun Zheng, Durham, NC (US); Stephen L. Craig, Durham, NC (US); Chenghao Duan, Durham, NC (US)

(21) Appl. No.: 18/883,403

(22) Filed: Sep. 12, 2024

Related U.S. Application Data
(60) Provisional application No. 63/537,879, filed on Sep. 12, 2023.

(51) Int. Cl. B01J 31/24; C07D 213/30; C07F 9/50

(52) U.S. Cl. CPC B01J 31/2409; C07D 213/30; C07F 9/5027; B01J 2231/643; B01J 2531/004; B01J 2531/16; B01J 2531/822

(57) ABSTRACT

Disclosed herein are systems and methods for modifying selectivity of heterogenous catalytic reactions.

### CROSS-REFERENCE TO RELATED APPLICATIONS

This application claims priority to and the benefit of U.S. Provisional Patent Application No. 63/537,879, filed on Sep. 12, 2023, the entire contents of which are incorporated herein by reference.

### STATEMENT REGARDING FEDERALLY SPONSORED RESEARCH

This invention was made with government support under DE-SC0018188 awarded by the Department of Energy. The government has certain rights in the invention.

### FIELD

Provided herein are systems and methods for modifying selectivity of heterogenous catalytic reactions.

### BACKGROUND

The selectivities of chemical reactions determine which value-added chemicals, pharmaceuticals, and polymers can be produced on a commercial scale, and they also determine the energy requirements and environmental impact associated with that production. The accessibility of a desired transformation is often improved through the use of a catalyst that lowers the energy barriers associated with a given reaction, and the development of improved catalysts is central to long-term economic and environmental sustainability.

Catalyst optimization for a given reaction usually involves changes in some combination of: the composition of the catalyst (e.g., choice of metal, ligand, or counter ion in transition metal catalyzed reactions) (see, e.g., Rechavi, et al. Chem. Rev. 102, 3467-3494 (2002); Tang et al. Chem. Rev. 103, 3029-3070 (2003)), temperature (see, e.g., Lautens et al. J. Am. Chem. Soc. 119, 11090-11091 (1997); Mohr et al. Nat. Chem. 1, 359-369 (2009)), pressure (see, e.g., Sun, et al. J. Am. Chem. Soc. 118, 1348-1353 (1996); Watkins et al. J. Am. Chem. Soc. 132, 10306-10317 (2010)), and solvent (see, e.g., Keck et al. J. Am. Chem. Soc. 117, 2363-2364 (1995); Cainelli et al. Chem. Soc. Rev. 38, 990-1001 (2009)) (FIG. 1A).

### SUMMARY

The present disclosure is based on the discovery of macroscopic, mechanical regulation of single-site catalytic selectivity, in which the extrinsic and symmetrical (achiral) mechanical stretching or swelling of a rubbery polymer support is delivered to a ligand-coordinated, single-site transition metal catalyst covalently coupled to that support (FIG. 1B). The macroscopic-to-molecular force transduction improves the selectivity (e.g., enantioselectivity) of the catalytic reaction and provides an approach to catalyst regulation that complements traditional methods. The enhancement increases with increasing strain, and the enantioselectivity returns to its nascent values once the strain is relaxed; the term “multi-state mechanocatalysis” (MMC) is used herein to reflect these attributes. Reactivity patterns across multiple substrates and force-coupling mechanisms support mechanochemical coupling as the unifying factor underlying the observed reactivity. Small molecule systems and computations were used to quantify the effect and elucidate mechanistic details. Although some recent reports have employed strained macrocyclic ligands (see, e.g., Kean et al. Angew. Chem. 126, 14736-14739 (2014); Wang et al. J. Am. Chem. Soc. 142, 17714-17720 (2020); Yu et al., Chem. Sci. 12, 11130-11137 (2021); Yu et al. ACS Catal. 12, 13941-13950 (2022); Duan et al. Organometallics 42, 1918-1926 (2023)), an approach using extrinsically modulated MMC has not been previously reported. Accordingly, relative to the few examples in which mechanical force has been used to convert a latent catalyst into an active one (Piermattei et al. Nat. Chem. 1, 133-137 (2009); Jakobs et al. Organometallics 31, 2476-2481 (2012); Groote et al. ACS Macro Lett. 1, 1012-1015 (2012); Groote, R., van Haandel et al. J. Polym. Sci., Part A: Polym. Chem. 50, 4929-4935 (2012); Michael et al. Angew. Chem., Int. Ed. 54, 13918-13922 (2015); Wei et al. ACS Macro Lett. 6, 1146-1150 (2017); Michael et al. Macromol. Rapid Commun. 39, 1800376 (2018)), MMC is distinguished by the potential it affords to toggle reversibly between multiple “on” states with different and tunable reaction outcomes.

Accordingly, in one aspect, disclosed herein is a method for conducting a catalytic reaction, comprising:
providing a catalyst covalently bound to a solid support;
conducting the catalytic reaction on the solid support; and
applying a force to the solid support to modify the selectivity of the catalytic reaction.

In some embodiments, the catalyst comprises a metal selected from rhodium, copper, palladium, nickel, platinum, ruthenium, iridium, and cobalt. In some embodiments, the metal is rhodium or copper.

In some embodiments, the catalyst comprises a ligand that is covalently bound to the solid support via at least two points of attachment. In some embodiments, the catalyst comprises a bidentate phosphine ligand having two reactive moieties that attach to the solid support.

In some embodiments, the ligand is a compound of formula (I):

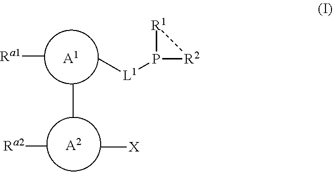

wherein:
A¹ and A² are each independently aryl or heteroaryl;
R^{a1} and R^{a2} are each independently selected from -Q-L-Y, wherein:
   Q is —OC(O)—, —NHC(O)—, or O;
   L is a bond or —(CH₂)ₙ—, wherein n is 1, 2, 3, 4, 5, or 6; and
   Y is a reactive group;

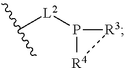

X is hydrogen or a group
L¹ and L² are each independently a bond or —CH₂—; and
R¹, R², R³, and R⁴ are each independently selected from C₁-C₆ alkyl, aryl, heteroaryl, and C₃-C₆ cycloalkyl, each of which is independently unsubstituted or substituted with 1 or 2 substituents, and wherein each dashed line represents the presence or absence of a covalent bond.

In some embodiments, the ligand is selected from:

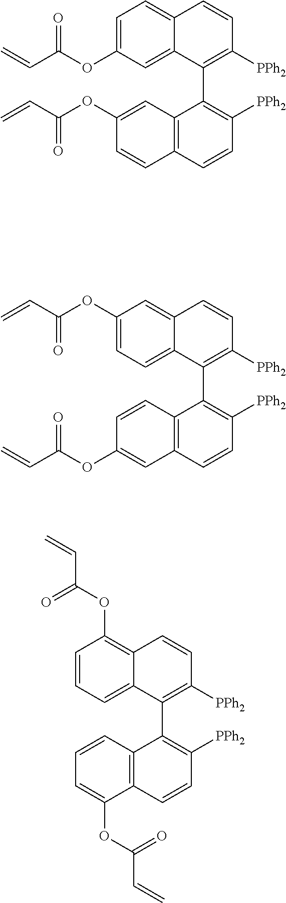

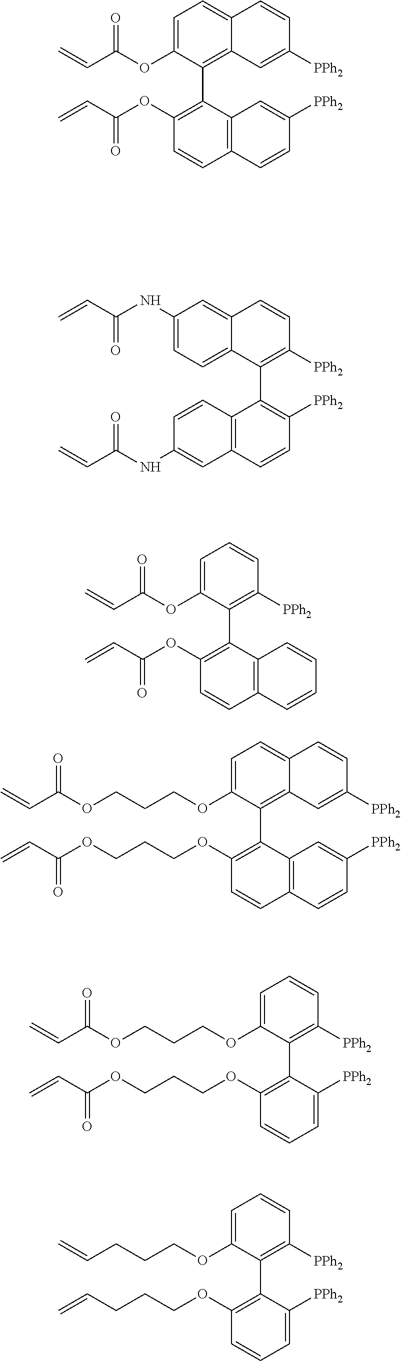

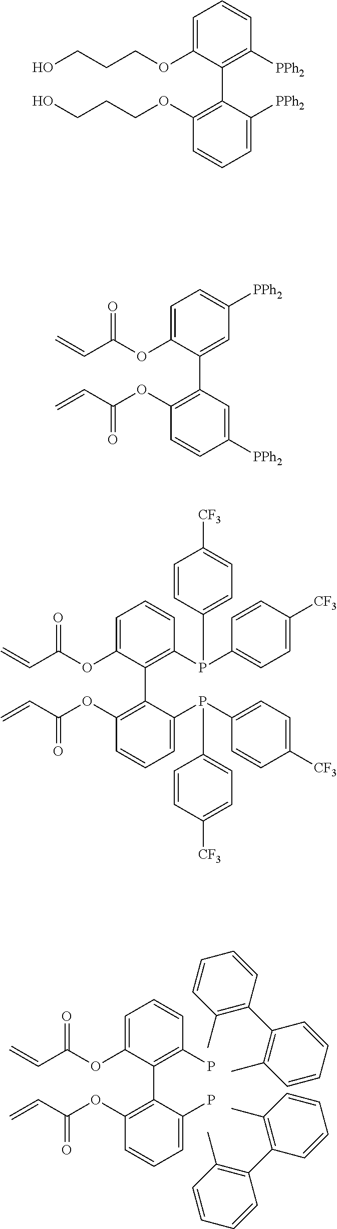

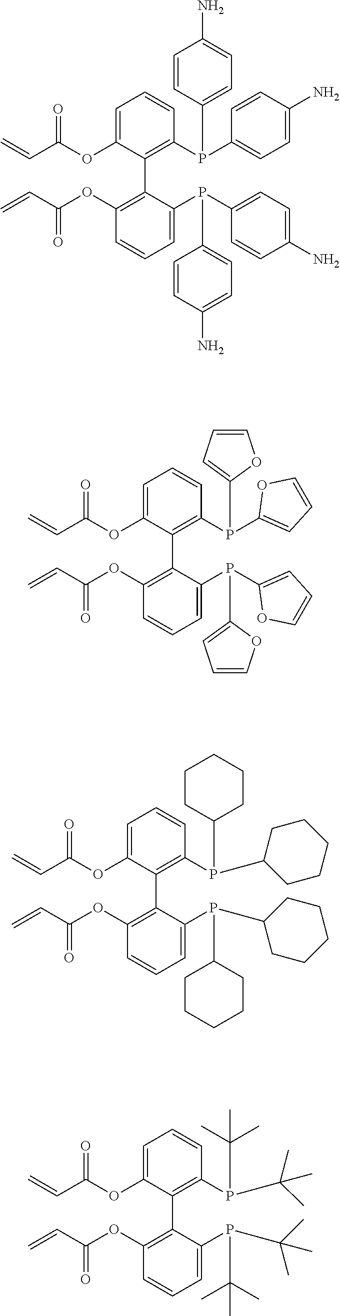 [SMILES: C=CC(=O)Oc1cccc(P(c2ccc(N)cc2)c2ccc(N)cc2)c1-c1c(OC(=O)C=C)cccc1P(c1ccc(N)cc1)c1ccc(N)cc1 ; C=CC(=O)Oc1cccc(P(c2ccco2)c2ccco2)c1-c1c(OC(=O)C=C)cccc1P(c1ccco1)c1ccco1 ; C=CC(=O)Oc1cccc(P(C2CCCCC2)C2CCCCC2)c1-c1c(OC(=O)C=C)cccc1P(C1CCCCC1)C1CCCCC1 ; C=CC(=O)Oc1cccc(P(C(C)(C)C)C(C)(C)C)c1-c1c(OC(=O)C=C)cccc1P(C(C)(C)C)C(C)(C)C]

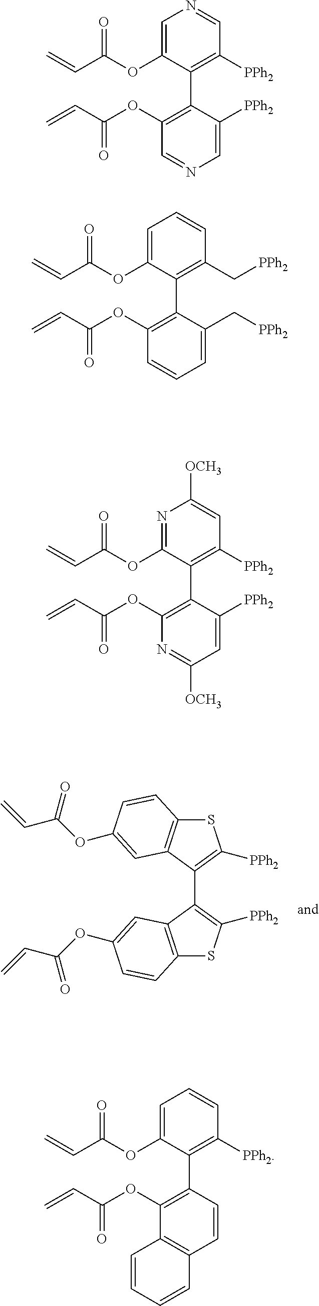

In some embodiments, the solid support is selected from single and double network organogels and hydrogels. In some embodiments, the solid support comprises a polymer network selected from polysiloxanes, polyurethanes, poly(meth)acrylates, and polyacrylamides. In some embodiments, the solid support comprises a polyacrylate polymer network. In some embodiments, the polyacrylate polymer network comprises a poly(alkyl acrylate). In some embodiments, the poly(alkyl)acrylate is selected from poly(methyl acrylate) and poly(ethyl acrylate).

In some embodiments, the catalytic reaction is selected from a hydrogenation reaction, a carbon-carbon coupling reaction, a hydroformylation reaction, a hydrosilylation reaction, an ethylene alkoxycarbonylation reaction, a hydroboration reaction, a carbonyl allylation reaction, a cycloaddition reaction, and a cyclotrimerization reaction. In some embodiments, the catalytic reaction is a hydrogenation reaction.

In some embodiments, the step of applying a force to the solid support comprises stretching the solid support. In some embodiments, the step of applying a force to the solid support comprises compressing the solid support. In some embodiments, the step of applying a force to the solid support comprises swelling the solid support. In some embodiments, swelling the solid support comprises contacting the solid support with a second solvent, wherein the second solvent is different from a first solvent used to carry out the catalytic reaction.

In some embodiments, the modification of the selectivity of the catalytic reaction comprises a modification of enantioselectivity, diastereoselectivity, and regioselectivity.

In another aspect, disclosed herein is a system for conducting a catalytic reaction, comprising:
(a) a solid support;
(b) a catalyst covalently bound to the solid support via at least two points of attachment;
wherein application of an external force to the solid support changes the selectivity of the catalytic reaction.

### BRIEF DESCRIPTION OF THE DRAWINGS

FIGS. 1A-1B show illustrations of approaches to improve selectivity in metal-catalyzed chemical reactions. FIG. 1A illustrates changing a ligand, metal, solvent, temperature, and/or pressure. FIG. 1B illustrates coupling of the catalyst complex to variable tension in a supporting polymer matrix.

FIGS. 2A-2D show partial GC (A and B) and HPLC (C and D) traces of the filtered reaction mixtures generated via hydrogenation of 1 (A), 3 (B), 4 (C), and 5 (D) catalyzed by a mixture of [Rh(COD)₂][BF₄] and bisphosphine ligand in MeOH at 25° C. for 24 h.

FIG. 3 shows ln(R/S) versus strain for the hydrogenation of 1 catalyzed by N2·Rh in MeOH at 25° C. subjected to uniaxial stretching

FIG. 4 shows ln(R/S) versus uniaxial compression for the hydrogenation of 1 catalyzed by N1·Rh in MeOH at 25° C.

FIG. 5 shows a calculated reaction mechanism at 0.5 and 2.0 nN.

FIG. 6 shows data from hydrogenation of methyl 2-acetamidoacrylate catalyzed by a molecular rhodium (I) complex. The overall reaction (equation (1)) of 1 produces a mixture of mirror image enantiomers of product 2. The free energy diagrams for the hydrogenation of 1 with (R)—Rh(MeOBiphep)⁺ at MN15/def2TZV level of the DFT in vacuum of the two diastereomeric paths with the structures associated with the pro-S pathway are depicted.

FIGS. 7A-7D show schematic representations of double networks for MMC and characterization data. FIG. 7A: .P_{act} was employed as a crosslinker in N1; FIG. 7B: P_{con} is employed as a co-monomer in N1_{con}. The cartoon shows how internal forces along stretched strands within N1 are coupled to the diaryl dihedral angle of the ligand in P_{act} but not P_{con}; FIG. 7C: UV-Vis absorption spectra of Rh(I)-free N1 gel, N1·Rh gel, and [(MeOBiphep)Rh(COD)]⁺BF₄⁻; FIG. 7D: Cyclic tensile experiment of Rh(I)-loaded N1·Rh gel.

FIGS. 8A-8D show a schematic of strained catalyst-containing materials and response of supported catalysts to straining. FIG. 8A: Schematic of network catalyst activity before, during, and after stretching; FIG. 8B: Fractional increase in er of hydrogenation by N1·Rh and N1_{con}·Rh under cyclic loading; FIG. 8C: Change in enantioselectivity of hydrogenation as a function of tensile strain across nine different batches of N1·Rh(denoted by numbered symbols). Entry 1 reproduces data shown in FIG. 8B; FIG. 8D: Change in enantioselectivity of hydrogenation as a function of compressive strain across two different batches of N1·Rh(denoted by numbered symbols). Uncertainty in strain is ±2%.

FIGS. 9A-9C show molecular force probe ligands and associated changes in enantioselectivity. FIG. 9A: Schematic representation of the compression (left) and extension (right) of a bisphosphine ligand in Z(m,n) and E(m,n) macrocycles; FIG. 9B: Measured enantiomeric ratios of reaction (1) as a function of the calculated force across the phenoxyl atoms. Error bars represent one standard deviation; FIG. 9C Enantiomeric ratios of reaction (1) at 9 values of tensile force applied across the _{MeO}C—C_{OMe} coordinate of the biphenyl ligand of the catalyst/substrate complex at the MN15/def2TZVP level of the DFT in vacuum; the values were derived by numerical kinetic modeling assuming irreversible traversal of TS2 (FIG. 6). It was confirmed that the constituent elementary steps of the reaction mechanism do not change at forces of 0.5 nN and 2.0 nN (FIG. 5) Lines connecting the circles are for guiding only.

FIGS. 10A-10C show force-coupled selectivity for other substrates. FIG. 10A: Force probe ligand effects relative to the MeOBiphep control; FIG. 10B: MMC of 3 due to compression of N1·Rh; FIG. 10C: Relative change in enantioselectivity of hydrogenation of 3 vs. 1 in different force-coupled settings.

FIG. 11 shows structures of molecular force probe ligands Z(m,n) and E(m,n).

FIG. 12 shows a plot of ln(er) versus restoring force for the copper-catalyzed hydrosilylation of acetophenone (Δ) and for the reductive coupling of 2-vinyl pyridine and acetophenone to form 2a (□) and 2b (O) (left), and a plot of ln(dr) versus restoring force for the copper-catalyzed reductive coupling of 2-vinyl pyridine and acetophenone (right).

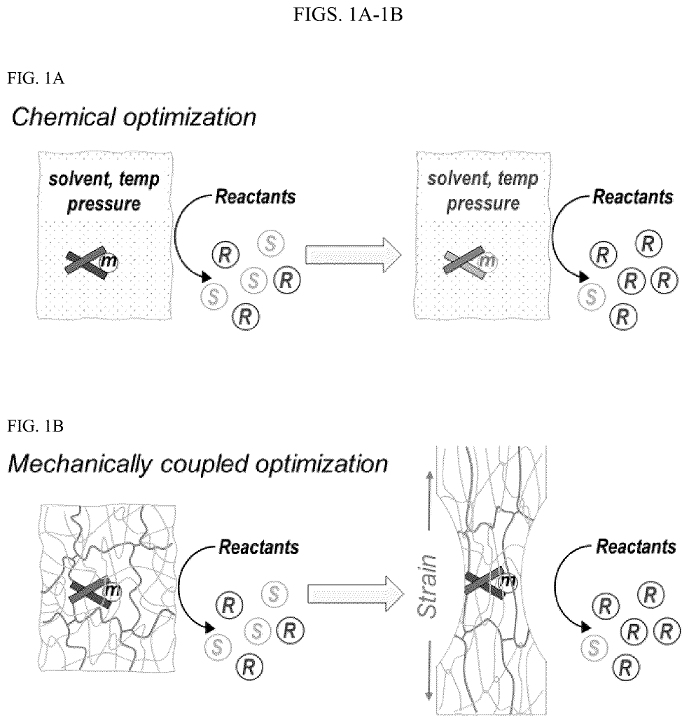

### DETAILED DESCRIPTION

Disclosed herein are methods and systems for modifying selectivity of heterogenous catalytic reactions. The methods and systems involve a catalyst bound to a solid support, wherein applying a force to the solid support, such as by stretching, compression, or swelling, modifies the selectivity of the catalytic reaction.

### Definitions

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein and in the appended claims, the singular forms “a”, “an”, and “the” include plural reference unless the context clearly dictates otherwise.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “amino” refers to a group —NR₂, wherein each R is independently selected from hydrogen and alkyl (e.g., C₁-C₄ alkyl). A group-NH (alkyl) may be referred to herein as “alkylamino” and a group-N(alkyl)₂ may be referred to herein as “dialkylamino.”

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl,” i.e., phenyl). In some embodiments, an aryl group has ten ring carbon atoms (“C₁₀ aryl,” e.g., naphthyl such as 1-naphthyl and 2-naphthyl). In some embodiments, an aryl group has fourteen ring carbon atoms (“C₁₄ aryl,” e.g., anthracenyl and phenanthrenyl).

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “halogen” or “halo” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroaryl, heterocyclyl, hydroxy, hydrazino, imino, oxo, nitro, phosphate, phosphonate, sulfonic acid, thiol, thione, or combinations thereof.

As used herein, the term “(meth)” designates optional methyl substitution. Thus, a term such as “(meth)acrylates” refers to both methacrylates and acrylates.

The polymer definitions are consistent with those disclosed in the Compendium of Polymer Terminology and Nomenclature, IUPAC Recommendations 2008, edited by: Richard G. Jones, Jaroslav Kahovec, Robert Stepto, Edward S. Wilks, Michael Hess, Tatsuki Kitayama, and W. Val Metanomski.

As used herein, a “polymer” is a macromolecule composed of the repeating units of the monomers used during polymerization.

As used herein, a “homopolymer” is a polymer made from one monomer; a “copolymer” is a polymer made from two or more monomers.

A “repeating unit” is the smallest group of atoms in a polymer that corresponds to the polymerization of a specific monomer or macromer.

An “initiator” is a molecule that can decompose into radicals which can subsequently react with a monomer to initiate a free radical polymerization reaction. For example photo-initiators decompose by a photochemical process; typical examples are derivatives of benzil, benzoin, acetophenone, benzophenone, camphorquinone, and mixtures thereof, as well as various monoacyl and bisacyl phosphine oxides and combinations thereof. Thermal initiators decompose at a certain rate depending on the temperature; typical examples are azo compounds such as 1,1′-azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems.

As used herein, a “crosslinker” or “cross-linking agent” is a di-functional or multi-functional monomer or macromer which can undergo free radical polymerization at two or more locations on the molecule, thereby creating branch points and a polymeric network. Common examples are ethylene glycol dimethacrylate, trimethylolpropane trimethacrylate, butane diacrylate, methylene bisacrylamide, triallyl cyanurate, and the like.

As used herein, in chemical structures the indication:

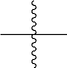

represents a point of attachment of one moiety to another moiety (e.g., a substituent group to the rest of the compound).

### Methods and Systems for Conducting Catalytic Reactions

Disclosed herein are methods and systems for conducting catalytic reactions, in which application of a force to a solid support having a catalyst covalently bound thereto modifies the selectivity of the catalytic reaction.

The methods and systems disclosed herein are used with a variety of different types of heterogenous metal-based catalysts. In particular, the methods and systems use a metal catalyst that is bound to a solid support, e.g., by at least two points of attachment.

The solid support typically includes a polymer network that is functionalized with a ligand, which in turn binds to the metal that serves as the active site for the catalytic reaction. The polymer network can be a single network (i.e., the polymer network includes a single polymer), or a double network (i.e., the polymer network includes two intertwined crosslinked polymer networks). In some embodiments, the polymer network is a hydrogel or an organogel. In the case of organogels, the organic solvent that forms the liquid phase of the organogel can be selected from the group consisting of methanol, ethanol, dichloromethane, toluene, methyl benzoate, ethyl acetate, dichlorobenzene, tetrahydrofuran, dimethylsulfoxide, and other solvents.

In some embodiments, the polymer that forms the polymer network is selected from polysiloxanes, polyurethanes, poly(meth)acrylates, and poly(meth)acrylamides. For example, in some embodiments, the polymer network comprises poly(dimethylsiloxane) (PDMS).

In some embodiments, the polymer network comprises one or more poly(meth)acrylates. For example, in some embodiments, the polymer network comprises at least one (meth)acrylate monomer selected from an alkyl (meth)acrylate, a hydroxyalkyl (meth)acrylate, an alkoxyalkyl (meth)acrylate, a cycloalkyl (meth)acrylate, and an aromatic (meth)acrylate. In some embodiments, the polymer network comprises an alkyl (meth)acrylate monomer selected from methyl (meth)acrylate, ethyl (meth)acrylate, n-propyl (meth)acrylate, n-butyl (meth)acrylate, iso-butyl (meth)acrylate, tert-butyl (meth)acrylate, n-hexyl (meth)acrylate, 2-ethylhexyl (meth)acrylate, octyl (meth)acrylate, iso-decyl (meth)acrylate, heptadecyl (meth)acrylate, dodecyl (meth)acrylate, 2-propylheptyl (meth)acrylate, and stearyl (meth)acrylate. In some embodiments, the polymer network comprises a hydroxyalkyl (meth)acrylate monomer selected from hydroxymethyl (meth)acrylate, 2-hydroxyethyl (meth)acrylate, 2-hydroxypropyl (meth)acrylate, 3-hydroxypropyl (meth)acrylate, 2,3-dihydroxypropyl (meth)acrylate, 2-hydroxybutyl (meth)acrylate. 3-hydroxybutyl (meth)acrylate, and 4-hydroxybutyl (meth)acrylate. In some embodiments, the polymer network comprises an alkoxyalkyl (meth)acrylate selected from 2-methoxyethyl (meth)acrylate, 2-ethoxyethyl (meth)acrylate, 1-methyl-2-methoxyethyl (meth)acrylate, ethylene glycol methyl ether (meth)acrylate, diethylene glycol methyl ether (meth)acrylate, and triethylene glycol methyl ether (meth)acrylate. In some embodiments, the polymer network comprises a cycloalkyl (meth)acrylate selected from cyclopentyl (meth)acrylate, cyclohexyl (meth)acrylate, 2-cyclohexylethyl (meth)acrylate, 3-cyclohexylpropyl (meth)acrylate, 2-norbornyl (meth)acrylate, and isobornyl (meth)acrylate. n some embodiments, the polymer network comprises a copolymer of at least two different (meth)acrylate monomers.

In some embodiments, the polymer network comprises a polyurethane, which is formed by reaction of a poly-isocyanate with a polyol.

In some embodiments, the polymer network comprises a poly(meth)acrylamide, such as polyacrylamide, poly(N-alkyl)acrylamides, and poly(N,N-dialkyl)acrylamides, in which the alkyl groups can be optionally substituted. Non-limiting examples of poly(meth)acrylamides include polyacrylamide, poly(N-isopropyl(meth)acrylamide), poly(N,N-dimethylaminopropyl(meth)acrylamide), poly(N,N-dimethyl(meth)acrylamide), poly(N-(2-hydroxyethyl)(meth)acrylamide), poly(N,N-bis(2-hydroxyethyl)(meth)acrylamide), poly(N-(2-hydroxypropyl)(meth)acrylamide), poly(N,N-bis(2-hydroxypropyl)(meth)acrylamide), poly(N-(3-hydroxypropyl)(meth)acrylamide), poly(N-(2-hydroxybutyl)(meth)acrylamide), poly(N-(3-hydroxybutyl)(meth)acrylamide), poly(N-(4-hydroxybutyl)(meth)acrylamide), poly(N-2-aminoethyl (meth)acrylamide), poly(N-3-aminopropyl (meth)acrylamide), poly(N-2-aminopropyl (meth)acrylamide), poly(N,N-bis-2-aminoethyl (meth)acrylamide), poly(N,N-bis-3-aminopropyl (meth)acrylamide), and poly(N,N-bis-2-aminopropyl (meth)acrylamide).

In some embodiments, the polymer network is a single polymer network comprising an alkyl (meth)acrylate, such as poly(ethyl acrylate). In some embodiments, the polymer network is a double polymer network, wherein one polymer network is prepared first, then is swollen and a second polymer network is synthesized. For example, in some embodiments, the polymer network is a double polymer network comprising two different alkyl (meth)acrylates, such as poly(ethyl acrylate) and poly(methyl acrylate).

The solid support has a catalyst covalently bound thereto, wherein the catalyst is covalently bound to the solid support (e.g., the polymer network) via at least two points of attachment. The catalyst is typically incorporated into the polymer network during the synthesis of the polymer network, by inclusion in the reaction mixture of a ligand having at least two reactive moieties that covalently link to the polymer network during the polymerization reaction. In some embodiments, the ligand is a monodentate or a bidentate ligand, such as a phosphine or a bisphosphine, having two reactive moieties such as (meth)acrylates, hydroxy groups, vinyl groups, or the like. The choice of reactive moiety will depend on the particular polymer network into which the ligand is being incorporated. For example, when the polymer network comprises poly(meth)acrylates, the reactive moieties can be (meth)acrylates. When the polymer network comprises a polyurethane, the reactive moieties can be hydroxy groups. When the polymer network comprises a polysiloxane, the reactive moieties can be vinyl groups, and the reaction mixture to form the polymer can include a vinyl-terminated polysiloxane (e.g., a vinyl-terminated poly(dimethylsiloxane).

In some embodiments, the ligand is a biaryl phosphine or a biaryl bisphosphine ligand. For example, in some embodiments, the ligand is a compound of formula (I):

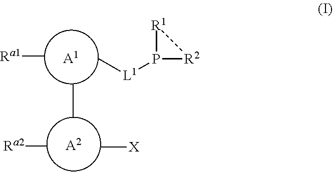

wherein:
A¹ and A² are each independently aryl or heteroaryl;
R^{a1} and R^{a2} are each independently selected from -Q-L-Y, wherein:
   Q is —OC(O)—, —NHC(O)—, or O;
   L is a bond or —(CH₂)ₙ—, wherein n is 1, 2, 3, 4, 5, or 6; and
   Y is a reactive group;

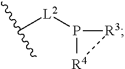

X is hydrogen or a group
L¹ and L² are each independently a bond or —CH₂—; and
R¹, R², R³, and R⁴ are each independently selected from C₁-C₆ alkyl, aryl, heteroaryl, and C₃-C₆ cycloalkyl, each of which is independently unsubstituted or substituted with 1 or 2 substituents, and wherein each dashed line represents the presence or absence of a covalent bond.

In some embodiments, A¹ and A² are each aryl. In some embodiments, A¹ and A² are each independently phenyl or naphthyl. In some embodiments, A¹ and A² are each a monocyclic or bicyclic heteroaryl having 1 heteroatom selected from N, S, and O. In some embodiments, A¹ and A² are each independently selected from pyridyl and benzothiophenyl.

In some embodiments, X is hydrogen. In some embodiments, X is a group

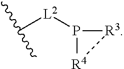

In some embodiments, L¹ and L² are each a bond. In some embodiments, L¹ and L² are each —CH₂—.

In some embodiments, R¹, R², R³, and R⁴ are each independently selected from C₁-C₆ alkyl, aryl, heteroaryl, and C₃-C₆ cycloalkyl.

In some embodiments, R¹, R², R³, and R⁴ are each aryl (e.g., phenyl). In some embodiments, R¹, R², R³, and R⁴ are each aryl (e.g., phenyl) that is unsubstituted or substituted with one substituent selected from C₁-C₄ alkyl, C₁-C₄ haloalkyl, C₁-C₄ alkoxy, and amino. In some embodiments, R¹, R², R³, and R⁴ are each phenyl that is unsubstituted or substituted with one substituent selected from methyl, trifluoromethyl, methoxy, or —NH₂. In some embodiments, R¹, R², R³, and R⁴ are each unsubstituted phenyl.

In some embodiments, the dashed lines between R¹ and R² and between R³ and R⁴ represent the absence of a bond. In some embodiments, the dashed lines between R¹ and R² and between R³ and R⁴ represent the presence of a bond. For example, in some embodiments, R¹ and R² are taken together to form a biphenyl, and R³ and R⁴ are taken together to form a biphenyl.

In some embodiments, R¹, R², R³, and R⁴ are each C₁-C₆ alkyl (e.g., tert-butyl). In some embodiments, R¹, R², R³, and R⁴ are each a monocyclic heteroaryl having 1 heteroatom selected from O, N, and S (e.g., furanyl). In some embodiments, R¹, R², R³, and R⁴ are each C₃-C₆ cycloalkyl (e.g., cyclohexyl).

In some embodiments, the ligand is a compound of formula (Ia):

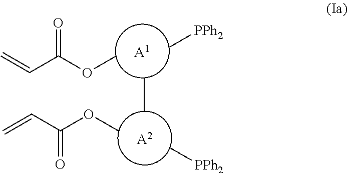

wherein A¹ and A² are as defined and described above.

In some embodiments, the ligand is selected from:

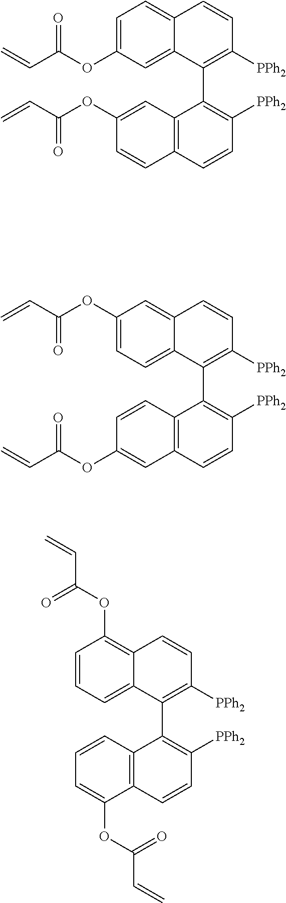

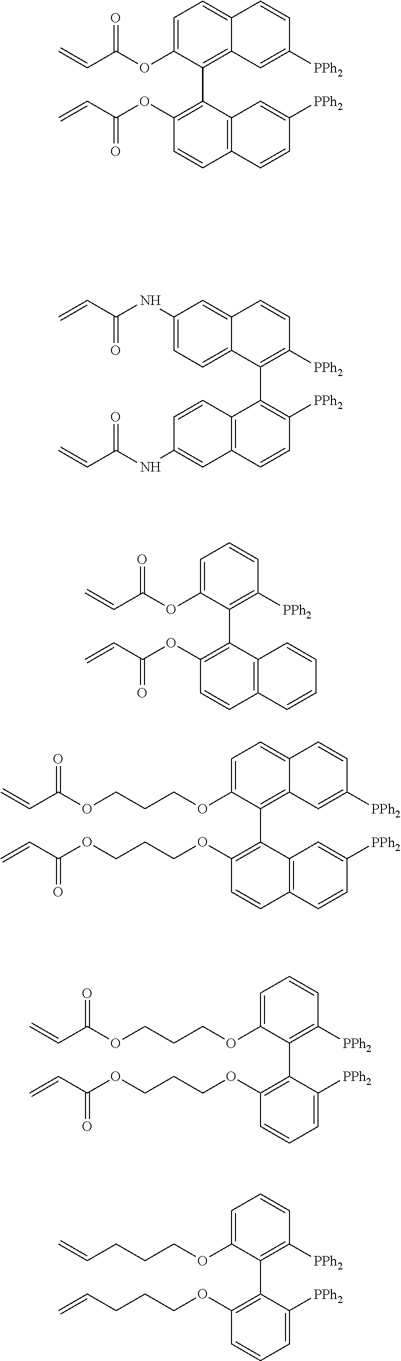

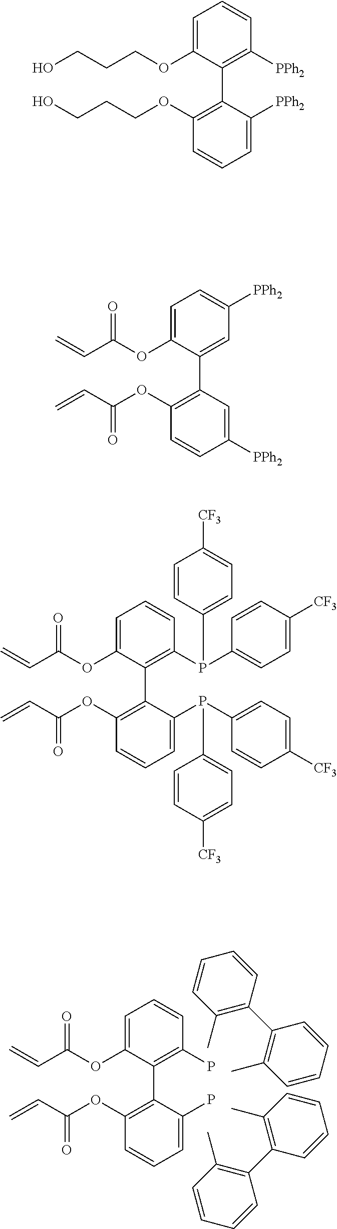

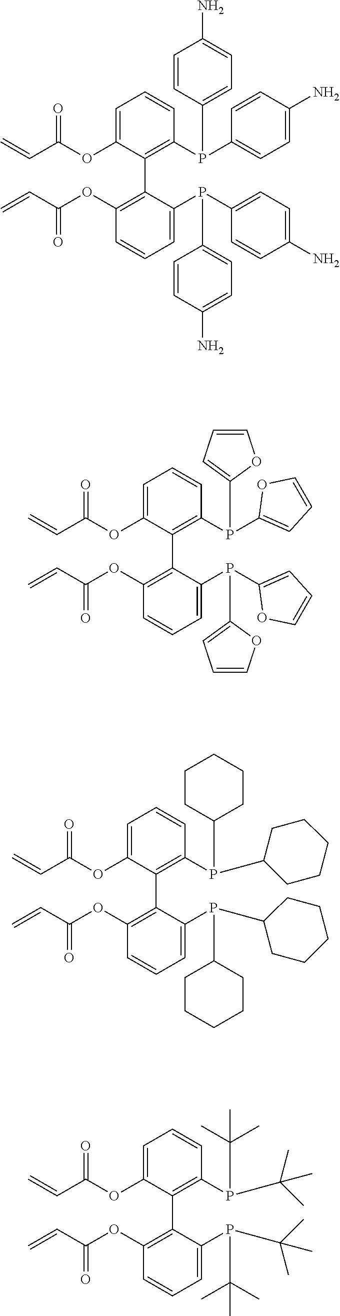 [SMILES: C=CC(=O)Oc1cccc(P(c2ccc(N)cc2)c2ccc(N)cc2)c1-c1c(OC(=O)C=C)cccc1P(c1ccc(N)cc1)c1ccc(N)cc1 ; C=CC(=O)Oc1cccc(P(c2ccco2)c2ccco2)c1-c1c(OC(=O)C=C)cccc1P(c1ccco1)c1ccco1 ; C=CC(=O)Oc1cccc(P(C2CCCCC2)C2CCCCC2)c1-c1c(OC(=O)C=C)cccc1P(C1CCCCC1)C1CCCCC1 ; C=CC(=O)Oc1cccc(P(C(C)(C)C)C(C)(C)C)c1-c1c(OC(=O)C=C)cccc1P(C(C)(C)C)C(C)(C)C]

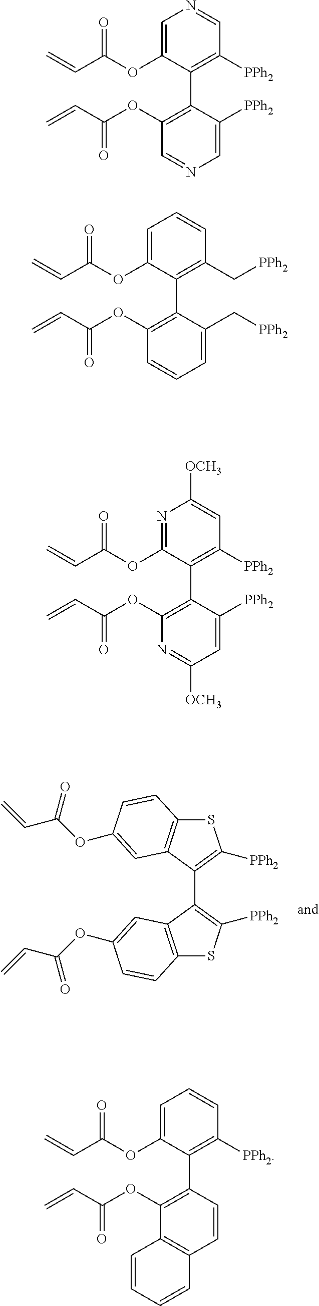

It will be apparent that the structures of the ligands shown above (including compounds of formula (I)) are of the ligands before they are incorporated into the polymeric network. After incorporation, the reactive moieties (e.g., acrylate group, hydroxy group, or vinyl group) will have reacted with monomers of the polymer to form cross-links to the polymer network, and thus the ligand will be covalently attached to the polymeric network. A representative structure is illustrated below:

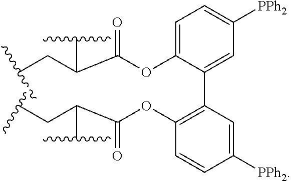

The ligands, having two points of attachment to the polymer network, will crosslink the polymer network during synthesis. In some embodiments, an additional crosslinker can be used during the polymerization reaction to further crosslink the polymer network. In some embodiments, the additional crosslinker is selected from methylene di(meth)acrylate, ethylene glycol di(meth)acrylate, propylene glycol di(meth)acrylate, butanediol di(meth)acrylate, diethylene glycol di(meth)acrylate, triethylene glycol di(meth)acrylate, tetraethylene glycol di(meth)acrylate, or the like. In some embodiments, the polymer network comprises a butanediol diacrylate crosslinker.

For synthesis of the polymer networks that form the solid support, monomers, the ligand, and an optional additional crosslinker can be combined in a solvent to form a reaction mixture, and a polymerization initiator may be added to the mixture. The polymerization initiator may include, for instance, at least one of lauroyl peroxide, benzoyl peroxide, iso propyl percarbonate, azobisisobutyronitrile, and the like, that generate free radicals at moderately elevated temperatures, or a photoinitiator system such as aromatic alpha-hydroxy ketones, alkoxyoxybenzoins, acetophenones, acylphosphine oxides, bisacylphosphine oxides, and a tertiary amine plus a diketone, mixtures thereof and the like. Illustrative examples of photoinitiators are 2-hydroxy-2-methylpropiophenone, 2-hydroxy-4′-(2-hydroxyethoxy)-2-methylpropiophenone, 1-hydroxycyclohexyl phenyl ketone, 2-hydroxy-2-methyl-1-phenyl-propan-1-one, bis(2,6-dimethoxybenzoyl)-2,4-4-trimethylpentyl phosphine oxide (DMBAPO), bis(2,4,6-trimethylbenzoyl)-phenyl phosphine oxide, 2,4,6-trimethylbenzyldiphenyl phosphine oxide and 2,4,6-trimethylbenzoyl diphenylphosphine oxide, benzoin methyl ester and a combination of camphorquinone and ethyl 4-(N,N-dimethylamino) benzoate. In some embodiments, the initiator is a photoinitiator, such as 2-hydroxy-2-methylpropiophenone. When photoinitiators are used, exposure to UV light initiates the polymerization process.

The ligand that is covalently bound to the polymer network binds to a metal atom that serves as the active site for catalysis. The metals can be incorporated into the polymer networks that have been functionalized with ligands after the polymerization reaction, e.g., by combining the polymeric network with a solution of a metal salt in order to load the metal ion into the polymer network. The particular metal salt can be a salt of any metal useful for conducting a catalytic reaction. For example, in some embodiments, the metal is selected from rhodium, copper, palladium, nickel, platinum, ruthenium, iridium, and cobalt. In some embodiments, the metal is rhodium. In some embodiments, the metal is copper.

Depending on the choice of metal, the catalytic reaction can be any of a number of reactions such as a hydrogenation reaction, a carbon-carbon coupling reaction, a hydroformylation reaction, a hydrosilylation reaction, an ethylene alkoxycarbonylation reaction, a hydroboration reaction, a carbonyl allylation reaction, a cycloaddition reaction, and a cyclotrimerization reaction. For example, in some embodiments, the reaction is a rhodium-catalyzed hydrogenation reaction (e.g., hydrogenation of alkenes or of α-ketoesters), a palladium-catalyzed cross-coupling reaction (e.g., a palladium-catalyzed Heck coupling reaction), a rhodium-catalyzed hydroformylation reaction, a copper-catalyzed hydrosilylation reaction, a copper-catalyzed reductive coupling reaction, a palladium-catalyzed ethylene alkoxycarbonylation reaction, a cobalt-catalyzed hydroboration reaction (e.g., a cobalt-catalyzed regioselective chain-walking hydroboration reaction), an iridium-catalyzed carbonyl allylation reaction, a cobalt-catalyzed Pauson-Khand reaction, a rhodium-catalyzed regioselective intermolecular cyclotrimerization reaction, or the like. In some embodiments, the metal is rhodium, and the catalytic reaction is a rhodium-catalyzed hydrogenation reaction. In some embodiments, the metal is copper, and the catalytic reaction is a copper-catalyzed reductive coupling reaction.

As demonstrated herein in the Examples, application of a force to the solid support can modify the selectivity of the catalytic reaction. For example, in some embodiments, the application of a force can modify the enantioselectivity, the diastereoselectivity, or the regioselectivity of the catalytic reaction. In some embodiments, the application of a force modifies the enantioselectivity of the catalytic reaction. In some embodiment, the selectivity (e.g., enantioselectivity, diastereoselectivity, or regioselectivity) is modified by at least 1%, at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, or at least 95%, or more, compared to the catalytic reaction conducted on the solid support without any force applied.

The application of force to the solid support can be carried out by stretching the solid support, compressing the solid support, or swelling the solid support. For example, in some embodiments, the step of applying a force to the solid support comprises stretching the solid support. In some embodiments, the step of stretching the solid support comprises stretching to a strain of about 1%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, or about 100%.

In some embodiments, the step of applying a force to the solid support comprises compressing the solid support. In some embodiments, the step of compressing the solid support comprises compressing the polymeric network by about 1%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, compared to the polymer network to which no force is applied.

In some embodiments, the step of applying a force to the solid support comprises swelling the solid support. The solid support can be swelled by exposing it to a different solvent. For example, in some embodiments, the step of applying a force to the solid support comprises contacting the solid support with a second solvent, wherein the second solvent is different from a first solvent used to carry out the catalytic reaction. In an exemplary embodiment, the first solvent is toluene and the second solvent is dichloromethane, and contacting the solid support with increasing amounts of dichloromethane increases the swelling ratio of the solid support.

As demonstrated in the examples, the modification in the selectivity of a catalytic reaction by application of a force to the solid support is reversible. Accordingly, following application of the force to modify the selectivity of the reaction, release of that force or return to the original state (e.g., by release of strain or by de-swelling) returns the selectivity of the catalytic reaction to a value at or near the original value, before any force was applied. This property allows the disclosed methods and systems to toggle reversibly between multiple “on” states, with different and tunable reaction outcomes.

### EXAMPLES

General Procedures. All reactions were performed under a nitrogen atmosphere in oven or flame-dried glassware employing standard Schlenk or glovebox techniques unless otherwise noted. All manipulations of single network, double network, and rhodium-loaded double network gels were performed in a nitrogen-filled glovebox. Nuclear magnetic resonance (NMR) spectra were obtained at 25° C. unless noted otherwise. ¹H and ¹³C chemical shifts are referenced to the solvent residual peaks, all the ¹³C NMR and ³¹P NMR spectra were proton decoupled, and ³¹P NMR spectra referenced using absolute frequency referencing in Mnova software. Gas chromatography (GC) was performed on a Shimadzu GCMS-QP2010 equipped with a Supelco β-Dex 225 (30 m×0.25 mm×0.25 μm) column using TIC detection. Chiral high performance liquid chromatography (HPLC) was performed on a Shimadzu Prominence Modular HPLC instrument using 4.6×250 mm columns. Anhydrous solvents were obtained either from Sigma-Aldrich in Sure/Seal™ containers or were dried and degassed using an Innovative Technologies PureSolv solvent purification system. All deuterated solvents were obtained from Cambridge Isotope Laboratory and were dried using activated 3 Å molecular sieves. All deuterated solvents were obtained from Cambridge Isotope Laboratory and were dried using activated 3 Å molecular sieves. All reagents and authentic samples of (R)-methyl acetylalaninate [(R)-2] and(S)-methyl acetylalaninate [(S)-2] were purchased from Sigma and were used as received unless otherwise noted. Methyl acrylate (MA), ethyl acrylate (EA), and butanediol diacrylate (BDA) were filtered through a column of neutral aluminum oxide prior to use. UV-initiated polymerizations were performed using a Vilbert-Lourmat UV-lamp (model VL-215.L). The polymerizations were initiated by 2-hydroxyethyl-2-methylpropiophenone (HMP). Macrocyclic bisphosphine ligands E/Z(m,n), intermediate I₁ and I₂ were synthesized according to published procedures (Kean 2014; Wang, et al. J. Org. Chem. 73, 5640-5642 (2008)).

### Example 1

### Synthesis of Bisphosphine Ligands

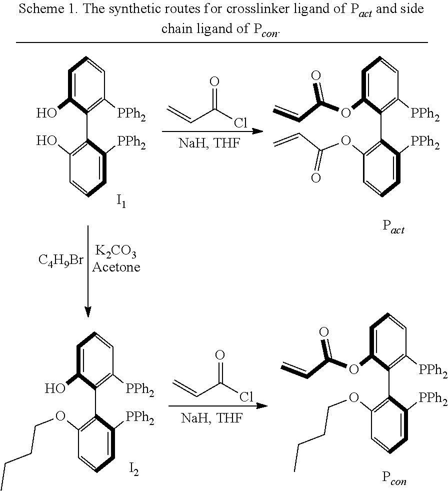

(R)-6,6′-Bis(diphenylphosphino)-[1,1′-biphenyl]-2,2′-diyl diacrylate (P_{act}). NaH (105.6 mg, 4.4 mmol) was added slowly to a solution of (R)-6,6′-bis (diphenylphosphinyl) biphenyl-2,2′-diol (I₁, 1.11 g, 2.0 mmol) in THF (10 mL) at 4° C. and the resulting suspension was stirred for 30 min. To this, acryloyl chloride (0.396 g, 4.4 mmol) was added dropwise and the resulting solution was stirred overnight at 4° C. The resulting solution was carefully quenched with ethanol (10 mL, 4° C.), and the solvent was evaporated under vacuum. The resulting oil was chromatographed (SiO₂: 0-50% EtOAc/hexane gradient) to give P_{act} (0.806 g, 60.8%) as a white solid. ¹H NMR (500 MHz, Chloroform-d): δ 7.32 (t, J=8.0 Hz, 2H), 7.29-7.19 (m, 20H), 7.17 (dd, J=8.0, 1.0 Hz, 2H), 7.06 (dq, J=8.0, 1.0 Hz, 2H), 5.93 (dd, J=17.0, 2.0 Hz, 2H), 5.69 (dd, J=17.0, 10.5 Hz, 2H), 5.63 (dd, J=10.5, 2 Hz, 2H). ¹³C{¹H} ¹NMR (126 MHz, Chloroform-d) δ 163.32, 148.76 (t, J=5.9 Hz), 140.25 (t, J=6.6 Hz), 137.9 (t, J=6.6 Hz), 136.2 (t, J=5.6 Hz), 134.55 (t, J=11.0 Hz), 134.1 (t, J=19.3 Hz), 133.09 (t, J=10.2 Hz), 131.89, 131.36, 128.75 (d, J=4.9 Hz), 128.28 (t, J=2.8 Hz), 128.20 (t, J=3.7 Hz), 128.05, 127.64, 122.56. ³¹P NMR (202 MHZ, Chloroform-d): δ−14.44. HRMS-ESI (m/z): [M+H]⁺ calculated for C₄₂H₃₃O₄P₂, 663.1854; observed, 663.1851.

(R)-2′-Butoxy-6,6′-bis(diphenylphosphaneyl)-[1,1′-biphenyl]-2-yl acrylate (P_{con}). NaH (39.6 mg, 1.65 mmol) was added slowly to a solution of (R)-2′-butoxy-6,6′-bis (diphenylphosphino)-[1,1′-biphenyl]-2-yl acrylate (I₂) (915 mg, 1.5 mmol) in THF at 4° C. and stirred for 30 min. To this solution, acryloyl chloride (149 mg, 1.65 mmol) was added dropwise and the resulting solution was stirred overnight at 0° C. After the reaction was completed, the system was carefully quenched by ethanol (10 mL, 4° C.), and the solvent was removed under reduced pressure. The resulting oil was chromatographed (0˜50% EtOAc/hexane gradient) to give P_{con} (840 mg, 84.3%) as a white solid. ¹H NMR (500 MHZ, Chloroform-d): δ 7.33-7.16 (m, 20H), 6.99 (ddd, J=7.5, 3.5, 1.5 Hz, 1H), 6.73 (ddd, J=7.5, 3.5, 1.0 Hz, 1H), 6.60 (dd, J=8.0, 1.0 Hz, 1H), 5.83 (dd, J=17.0, 1.5 Hz, 1H), 5.72 (dd, J=17.0, 10.0 Hz, 1H), 5.59 (dd, J=10.0, 1.5 Hz, 1H), 3.45 (ddd, J=9.0, 7.5, 6.5 Hz, 1H), 3.16 (ddd, J=9.0, 8.0, 5.5 Hz, 1H), 1.31-1.15 (m, 3H), 1.13-0.99 (m, 2H), 0.76 (t, J=7.5 Hz, 3H). ¹³C{¹H} NMR (126 MHz, Chloroform-d): δ 163.01, 156.86 (d, J=10.5 Hz), 148.99 (d, J=9.2 Hz), 140.05 (d, J=9.3 Hz), 139.26 (d, J=9.2 Hz), 138.40 (dd, J=16.3, 13.4 Hz), 136.88 (dd, J=23.8, 13.1 Hz), 134.55 (d, J=8.5 Hz), 134.39 (d, J=8.3 Hz), 133.32 (dd, J=13.6, 3.4 Hz), 133.18 (dd, J=13.1, 2.8 Hz), 131.15, 130.91, 128.96, 128.45, 128.25, 128.19 (d, J=4.6 Hz), 128.13, 128.02, 127.97, 127.80 (d, J=7.3 Hz), 125.53, 122.18, 110.88, 67.19, 30.60, 18.77, 13.79. ³¹P NMR (202 MHz, Chloroform-d): δ−13.05 (d, J=42.4 Hz), −13.67 (d, J=42.4 Hz). HRMS-ESI (m/z): [M+H]⁺ calculated for C₄₃H₃₉O₃P₂, 665.2374; observed, 665.2366.

### Example 2

### Rh-Catalyzed Hydrogenation Employing Molecular Catalysts

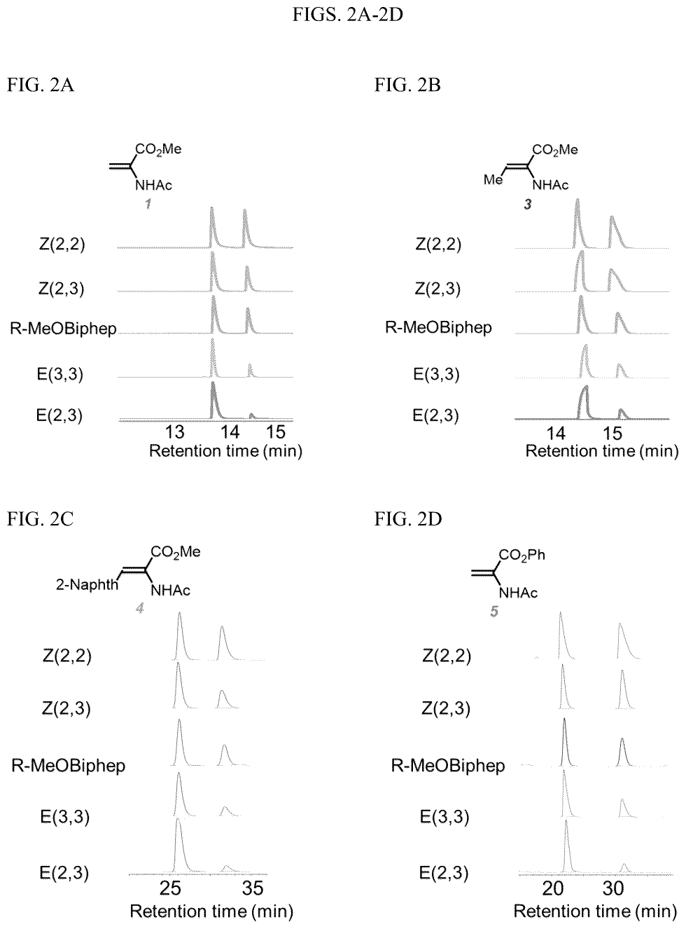

Effect of restoring force on enantioselectivity. A solution of [Rh(COD)₂][BF₄] (0.508 mg, 1.25 μmol, 2.5 μM) and P—P [P—P=MeOBiphep, Z(2,2), Z(2,3), E(2,3), E(3,3); 2.5 μmol, 5.0 μM) in MeOH (0.5 mL) in a 5 mL vial was stirred at 25° C. for 15 min. The resulting solution was added to a solution of 2-amidoacrylate (1, 17.9 mg, 125 μmol, 250 μM) in MeOH (0.5 mL). The vial was capped with a septum and connected to a hydrogen-filled balloon via a needle and the solution was stirred (300 rpm) at 25° C. for 24 h. The resulting solution was passed through a plug of silica gel and analyzed by GC equipped with a Supelco β-Dex 225 column (30 m×0.25 mm×0.25 μm) employing an initial oven temperature of 70° C. for 3.0 min, followed by a temperature increase to 140° C. at a rate of 5.0° C./min, and then to 215° C. at a rate of 15.0° C./min. The relative concentrations of 1, (R)-2, and(S)-2 were determined by integrating the GC peaks at 12.7 min, 13.7 min, and 14.4 min, respectively (FIG. 2A, Table 1). CG peak assignments were confirmed using authentic samples of 1, (R)-2, and(S)-2. The effect of restoring force on the enantioselective hydrogenation of 2-acetamidoacrylates 3 (Chen et al. Org. Lett. 20 (11), 3278-3281), 4 (Jiang et al. J. Am. Chem. Soc. 2020, 142, 9642-9652), and 5 (Obara et al. Synlett, 28, 1183-1186) were determined employing analogous procedures (Table 1). For the hydrogenation of 3, the filtered reaction mixture was analyzed by GC equipped with a Supelco β-Dex 225 column (30 m×0.25 mm×0.25 μm). The relative concentrations of R and S methyl 2-acetamidobutanoate were determined by integrating the peaks at t_{R}=14.5 min and t_{R}=15.1 min, respectively (FIG. 2B). For the hydrogenations of 4 and 5, the filtered reaction mixture was analyzed by HPLC equipped with a Chiralcel OD-H column (90:10 hexane: i-PrOH, 0.5 mL/min, 25° C., 220 nm). The relative concentrations of R and S methyl 2-acetamido-3-(naphthalen-2-yl) propanoate were determined by integrating the peaks at t_{R}=18.9 min and t_{R}=25.6 min, respectively (FIG. 2C). The relative concentrations of D and L phenyl acetylalaninate were determined by integrating the peaks at t_{R}=21.5 min and t_{R}=31.5 min, respectively (FIG. 2D).

TABLE 1
Enantioselectivity of the Rh(I)-Catalyzed Hydrogenation of 1, 3, 4, and 5 as a
Function of Ligand Restoring Force in MeOH at 25° C. under H₂ (1 atm) for 24 h.

| | | 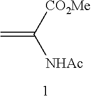 | 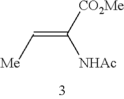 [SMILES: C/C=C(\NC(C)=O)C(=O)OC] | 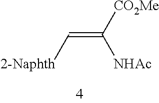 | 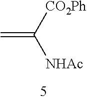 |
|---|---|---|---|---|---|
| est. force (pN) | MFPs | ee (%)^{a} | ee (%)^{a} | ee (%)^{b} | ee (%)^{b} |
| −65 | Z(2,2) | 2 | 1 | 9 | −6 |
| −6 | Z(2,3) | 28 | 13 | 36 | −4 |
| 0 | (R)—MeOBiphep | 26 | 18 | 32 | 10 |
| 130 | E(3,3) | 54 | 39 | 62 | 38 |
| 228 | E(2,3) | 84 | 65 | 82 | 70 |

^{a}analyzed by GC;
^{b}analyzed by HPLC

Enantioselectivity as a function of conversion. A solution of [Rh(COD)₂][BF₄] (0.508 mg, 1.25 mmol, 2.5 mM) and Z(2,3) (2.2 mg, 2.5 mmol, 5.0 mM) in MeOH (0.5 mL) was stirred under N2 (1 atm) at 25° C. for 15 min in a septum-capped 5 mL vial equipped with a magnetic stir bar and then under H₂ (balloon) for 30 min. The resulting activated catalyst solution was added to a solution of 2-amidoacrylate (1, 17.9 mg, 125 mmol, 250 mM) in MeOH (0.5 mL), the system was recharged with hydrogen (balloon), stirred (300 rpm) at 25° C., and monitored periodically by GC (Table 2).

TABLE 2
Enantioselectivity of Rh(I)-catalyzed reaction of 1 with
H₂ (1 atm) catalyzed by a mixture of [Rh(COD)₂][BF₄]
and Z(2,3) in methanol at 25° C. as a function of conversion.

| time (min) | conversion (%) | er |
|---|---|---|
| 0 | 0 | — |
| 2 | 46 | 64.0:36.0 |
| 4 | 62 | 64.5:35.5 |
| 6 | 76 | 64.4:35.6 |
| 10 | 90 | 64.5:35.5 |
| 18 | 99 | 64.1:35.9 |

Effect of P—P/Rh concentration on enantioselectivity. A solution of [Rh(COD)₂][BF₄] (0.31-1.2 mM) and bisphosphine ligand (0.31-1.2 mM) in MeOH (0.5 mL) was stirred under N2 (1 atm) at 25° C. for 15 min in a septum-capped 5 mL vial. The vial was connected to a H₂ filled balloon via a needle and the solution was stirred for an additional 30 min. The resulting activated catalyst solution was added to a solution of 2-amidoacrylate (1, 17.9 mg, 125 μmol, 250 μM) in MeOH (0.5 mL) in a septum-capped 5 mL vial equipped with a magnetic stir bar. The vial was connected to a H₂ filled balloon via a needle and the solution was stirred (300 rpm) at 25° C. for 6-90 minutes (Table 3).

TABLE 3
Enantioselectivity of Rh(I)-catalyzed reaction of 1 (17.9
mg, 125 μmol, 125 μM) with H₂ (1 atm) catalyzed by a mixture of
[Rh(COD)₂][BF₄] and Z(2,3) in methanol at 25°
C. as a function of rhodium and ligand concentration.

| [Rh(COD)₂][BF₄] | Z(2,3) | time | conversion | |
|---|---|---|---|---|
| (μM) | (μM) | (min) | (%) | er |
| 0.313 | 0.313 | 90 | 21 | 63.8:36.2 |
| 0.625 | 0.625 | 80 | 99 | 64.1:35.9 |
| 1.25 | 1.25 | 6 | 99 | 63.8:36.2 |
| 1.25 | 2.5 | 18 | 99 | 64.4:35.7 |

Effect of H₂ pressure on enantioselectivity. A solution of [Rh(COD)₂][BF₄] (5.075 mg, 12.5 μmol, 25 mM) and MeOBiphep (14.56 mg, 25 μmol, 50 μM) in MeOH (5 mL) in a 20 mL vial was stirred at 25° C. for 15 min. The mixture was then stirred under H₂ (balloon) for 30 min. The activated catalyst solution (0.5 mL, 1 mol %) was added to a solution of 2-amidoacrylate (1, 17.9 mg, 125 μmol, 125 μM) in MeOH (0.5 mL) in a thick-wall flask equipped with a magnetic stir bar. The flask was connected to a hydrogen tank fitted with a pressure regulator, the flask was pressurized with H₂, and the solution was stirred at 300 rpm for 5 min. The flask was depressurized and the resulting solution was analyzed by GC (Table 4).

TABLE 4
Hydrogenation of methyl 2-acetamidoacrylate (1.46 mg,
2.50 μmol, 2.50 μM) using MeOBiphep (1.46 mg, 2.50
μmol, 2.50 μM) as the ligand and Rh(I) (0.508 mg, 1.25
μmol, 1.25 μM) as metal as a function of H₂ pressure
for 5 min in MeOH (1 mL) at 25° C.

| p(H₂) (psi) | yield (%) | R:S |
|---|---|---|
| 10 | 27.9 | 63.1:36.9 |
| 15 | 47.9 | 63.4:36.6 |
| 20 | 99.3 | 63.5:36.5 |
| 30 | 100 | 63.8:36.2 |
| 40 | 100 | 64.0:36.0 |
| 50 | 100 | 64.2:35.8 |

### Example 3

### Network Synthesis

### Synthesis of First Networks

S1. A 50% (w/w) solution of ethyl acrylate (EA) as monomer, P_{act} (2.5 mol % of monomer) as crosslinker, and 2-hydroxy-2-methylpropiophenone (HMP) (1 mol % of monomer) as initiator in toluene was sonicated for 10 min and then slowly poured into a glass mold consisting of two flat soda-lime glass plates (thickness: 3 mm) separated by silicone rubber (thickness: 0.8 mm) as a spacer. The solutions were irradiated with UV light (365 nm, 4 mW·cm⁻²) from both sides of the glass mold for 24 h. The samples were then removed from the mold and immersed in toluene for at least a week to wash out unreacted components, and dialysis baths were changed every day. The resulting swollen first network S1 was subsequently used for double network synthesis. The reactant feed concentrations are summarized in Table 5.

S1_{con}. A 50% (w/w) solution of EA/P_{con} (97.5/2.5 molar ratio) as co-monomers, butanediol diacrylate (BDA, 2.5 mol % of monomers) as crosslinker, and HMP (1 mol % of monomers) as initiator in toluene was sonicated for 10 min and then slowly poured into a glass mold consisting of two flat soda-lime glass plates (thickness: 3 mm) separated by silicone rubber (thickness: 0.8 mm) as a spacer. The solutions were irradiated with UV light (365 nm, 4 mW·cm⁻²) from both sides of the glass mold for 24 h. The samples were then removed from the mold and immersed in toluene for >1 week to wash out unreacted components with dialysis baths changed daily. The resulting swollen first network S1con was subsequently used for double network synthesis. The reactant concentrations are summarized in Table 5.

S2. A 50% solution (w/w) of EA as monomer, P_{act} (1.0 mol % of monomer) as crosslinker, and HMP (1 mol % of monomer) as initiator was sonicated for 10 min and then slowly poured into a glass mold consisting of two flat soda-lime glass plates (thickness: 3 mm) separated by silicone rubber (thickness: 0.8 mm) as a spacer. The solutions were irradiated with UV light (365 nm, 4 mW·cm⁻²) from both sides of the glass mold for 24 h. The samples were then extracted from the mold and immersed in toluene for ≥1 week with dialysis baths changed daily. The resulting swollen first network S2 was subsequently used for double network synthesis. The reactant concentrations are summarized in Table 5.

TABLE 5
Feed composition of single network gels in toluene (1.0 g).

| | EA | P_{con} | P_{act} | BDA | HMP | Cured SN |
|---|---|---|---|---|---|---|
| network | (mg/mmol) | (mg/mmol) | (mg/mmol) | (mg/mmol) | (mg/mmol) | mass (mg) |
| S1 | 1000/10 | — | 165.5/0.25 | — | 16.4/0.1 | 960 |
| S1_{con} | 975/9.75 | 166/0.25 | — | 49.5/0.25 | 16.4/0.1 | 1000 |
| S2 | 1000/10 | — | 66.2/0.1 | — | 16.4/0.1 | 860 |

### Synthesis of Double Networks

The feed composition of the second networks used in the syntheses of double networks N1, N1_{con}, and N2 are identical. The dialyzed first networks were swollen in a solution of methyl acrylate (MA, 100 g) as second monomer, BDA (57.5 mg, 0.025 mol % of monomer) as crosslinker, and HMP (9.5 g, 0.005 mol % of monomer) as UV initiator for 3 days. The swollen gels were then sandwiched between two flat soda-lime glass plates and irradiated with UV light (365 nm, 4 mW·cm⁻²) for 24 h to synthesize the stretchable second PolyMA network in the presence of the brittle first networks. The double network gels were then removed from the mold and immersed in toluene for ≥1 week with dialysis baths changed daily, dried under vacuum at 25° C. for 3 days, and stored at room temperature until later use. The compositions of the second network of double network gels are summarized in Table 6. The concentration of bisphosphine ligand in the resulting DNS (Table 6) was estimated employing equation S1, where C_{P—P} denotes the concentration of ligand in the resulting cured DN; M_{cured} denotes the mass of cured SN gel; Mₜₒₜₐₗ denotes the total mass of the feed monomers employed in the synthesis of the SN; M_{L-feed} denotes the molar amount of bisphosphine (P_{act} or P_{con}) employed in the synthesis of the SN, and M_{DN} denotes the mass of the cured DN.

$${C}_{P - P} ( m mol / g ) = [ ( {M}_{c u r e d} ( g ) / {M}_{total} ( g ) ) \times {M}_{L - feed} ] / {M}_{D N} ( g ) \qquad eq S1$$

TABLE 6
Composition of second network of double network gels.

| | MA | BDA (mg, | HMP (mg, | Cured | P-P conc. |
|---|---|---|---|---|---|
| network | (g/mol) | mmol) | mmol) | network (g) | (mmol/g) |
| N1 | 100, 1.16 | 57.5, 0.29 | 9.5, 0.116 | 4.6 | 4.6 × 10⁻² |
| N1_{con} | 100, 1.16 | 57.5, 0.29 | 9.5, 0.116 | 4.4 | 4.8 × 10⁻² |
| N2 | 100, 1.16 | 57.5, 0.29 | 9.5, 0.116 | 5.5 | 1.5 × 10⁻² |

Loading rhodium to DNs. Excised samples of double networks N1 (˜50 mg, ˜2.3 μmol bisphosphine), N1_{con} (˜50 mg, ˜3.0 μmol bisphosphine), and N2 (˜50 mg, ˜3.0 μmol bisphosphine) were immersed in solutions of [Rh(COD)₂][BF₄] (2.0 equiv. relative to bisphosphine) in MeOH (2 mL) for ≥3 days at 25° C. The resulting Rh(I)-loaded DNs N1·Rh, N1_{con}·Rh, and N2·Rh were then immersed in MeOH (2 mL) for ≥3 days to remove unbound Rh with the baths changed every 12 h. The resulting Rh(I)-loaded DN catalyst gels were then employed for subsequent hydrogenation and characterization.

Cyclic testing of N1·Rh. Uniaxial tensile cyclic testing of N1·Rh was performed at the nominal strain rate of 1%/s. The samples were stretched to a target strain (50, 60, 70, and 100%) and then immediately unloaded at the same rate. The nominal strain (8) is given by Eq S2. The nominal stress was calculated as the measured load divided by the original cross-sectional area vertical to the load.

$$ε = ( {L}_{c} - {L}_{i} ) / {L}_{i} , where {L}_{c} = stretched length and {L}_{i} = initial length \qquad ( eq S 2 )$$

### Example 4

### Rh-Catalyzed Hydrogenation on Solid Supports

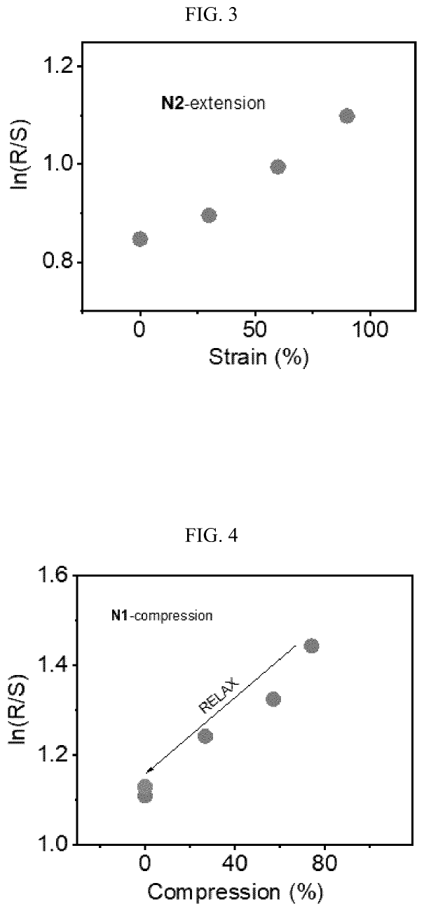

Uniaxial stretching. Catalytic hydrogenation of 1 employing rhodium-loaded DNs as a function of uniaxial strain were performed employing a bespoke stretching device of approximate dimensions 5.5×2.1×2.1 cm comprising a pair of 3 mm thick clamps positioned 6 mm apart with one clamp anchored to the base of the device and the second anchored to a movable platform. The platform is connected to a thumb screw such that turning the thumb screw increases the separation of the clamps with up to 16 mm separation (167% strain) at full travel. A sample of N1·Rh(˜3 mm×˜12 mm×˜1.1 mm; 50 mg, 2.3 μmol Rh) was securely clamped onto the stretching device. The secured gel and device were then placed in a 40 mL vial containing a solution of 2-amidoacrylate (1, 3.3 mg, 3.8 mM) in MeOH (6 mL), which was sufficient volume to completely submerge the gel. The vial was capped with a septum, attached to a hydrogen-filled balloon via a needle, and the liquid phase was stirred at 25° C. for 2 days and then analyzed by GC (Table 7, entry 1). Without removing the gel from the mold, N1·Rh was rinsed three times with methanol, strained at 22%, and immersed in a fresh solution of 1 (3.3 mg, 3.8 mM) in MeOH (6 mL) in a 40 mL vial; percent strain was determined by measuring the distance between the clamps before and after the gel was stretched using a caliper. The vial was again capped with a septum and attached to a hydrogen-filled balloon via a needle. The liquid phase was stirred at 25° C. for 2 days and then analyzed by GC (Table 7, entry 2). The procedure was then repeated sequentially at 40%, 50%, and 0% strain (Table 7, entries 3-5). Catalytic hydrogenation of 1 employing eight additional batches of N1·Rh and N1_{con}·Rh and N2·Rh as a function of uniaxial strain were performed employing a similar procedure (Table 7, entries 6-31, FIG. 3).

TABLE 7
Enantioselective hydrogenation of 1 catalyzed by solid
supports N1•Rh, N1_{con}•Rh, and N2•Rh in methanol under
H₂ (1 atm) at 25° C. as a function of uniaxial stretching.

| | | | | time | | |
|---|---|---|---|---|---|---|
| entry | support | sample | e (%) | (days) | yield (%) | er (R:S) |
| 1 | N1•Rh | 1 | 0 | 2 | 77 | 3.00 |
| 2 | N1•Rh | 1 | 22 | 2 | 34 | 3.26 |
| 3 | N1•Rh | 1 | 40 | 2 | 21 | 3.88 |
| 4 | N1•Rh | 1 | 50 | 4 | 44 | 4.26 |
| 5 | N1•Rh | 1 | 0 | 4 | 31 | 3.00 |
| 6 | N1•Rh | 2 | 0 | 2 | 66 | 3.14 |
| 7 | N1•Rh | 2 | 50 | 2 | 44 | 3.93 |
| 8 | N1•Rh | 3 | 0 | 2 | 96 | 3.17 |
| 9 | N1•Rh | 3 | 50 | 2 | 64 | 3.81 |
| 10 | N1•Rh | 4 | 0 | 2 | 78 | 3.24 |
| 11 | N1•Rh | 4 | 50 | 2 | 32 | 4.31 |
| 12 | N1•Rh | 5 | 0 | 2 | 97 | 3.04 |
| 13 | N1•Rh | 5 | 30 | 2 | 40 | 3.39 |
| 14 | N1•Rh | 5 | 53 | 2 | 43 | 3.95 |
| 15 | N1•Rh | 6 | 0 | 2 | 71 | 2.58 |
| 16 | N1•Rh | 6 | 30 | 2 | 62 | 2.97 |
| 17 | N1•Rh | 6 | 60 | 2 | 30 | 3.37 |
| 18 | N1•Rh | 7 | 0 | 2 | 70 | 3.09 |
| 19 | N1•Rh | 7 | 50 | 2 | 44 | 3.78 |
| 20 | N1•Rh | 8 | 0 | 2 | 89 | 3.00 |
| 21 | N1•Rh | 8 | 50 | 2 | 78 | 3.90 |
| 22 | N1•Rh | 9 | 0 | 2 | 95 | 3.16 |
| 23 | N1•Rh | 9 | 50 | 2 | 66 | 3.88 |
| 24 | N1_{con}•Rh | — | 0 | 2 | 42 | 1.74 |
| 25 | N1_{con}•Rh | — | 32 | 2 | 28 | 1.78 |
| 26 | N1_{con}•Rh | — | 62 | 2 | 17 | 1.78 |
| 27 | N1_{con}•Rh | — | 0 | 2 | 13 | 1.74 |
| 28 | N2•Rh | — | 0 | 2 | 37 | 2.28 |
| 29 | N2•Rh | — | 30 | 2 | 12 | 2.42 |
| 30 | N2•Rh | — | 60 | 2 | 18 | 2.82 |
| 31 | N2•Rh | — | 90 | 2 | 7 | 3.07 |

Uniaxial compression. Catalytic hydrogenation of 1 employing Rh-loaded DNs as a function of uniaxial compression were performed employing a compressing device comprising two 21 mm diameter stainless steel plates attached to one another via four machine screws which control the separation of the plates. Rh(I)-loaded DN N1·Rh of dimensions ˜6 mm×˜6 mm×˜1.1 mm (˜92 mg, 4.2 μmol Rh) was secured between the plates of the compressing device and the secured gel and device were then immersed in a solution of methyl 2-amidoacrylate (1, 6 mg, 21 mM) in MeOH (2 mL) in a 40 mL vial. The vial was capped with a septum and attached to a hydrogen-filled balloon via a needle. The liquid phase was stirred at 25° C. for 2 days and then analyzed by GC (Table 8, entry 1). The sample of N1·Rh was rinsed three times with methanol, compressed 27% by measuring the distance between the plates before and after compression using a caliper, and immersed in a fresh solution of 1 in MeOH in a 40 mL vial under H₂ (balloon). The solution phase was stirred for 2 days and analyzed by GC (Table 8, entry 2). The procedure was then repeated at 57%, 74%, and 0% compression (Table 8, entries 3-5). The enantioselective hydrogenations of 1 and 3 as a function of uniaxial compression employing a separate batch sample of N1·Rh was performed employing a similar procedure (Table 9). Data are also shown in FIG. 4.

TABLE 8
Enantioselective hydrogenation of 1 catalyzed by
N1•Rh in methanol under H₂ (1 atm) at 25°
C. as a function of uniaxial compression.

| entry | compression (%) | yield (%) | time (days) | er |
|---|---|---|---|---|
| 1 | 0 | 17.1 | 2 | 3.03 |
| 2 | 27 | 25.7 | 3 | 3.46 |
| 3 | 57 | 3.5 | 4 | 3.76 |
| 4 | 74 | 6.5 | 8 | 4.24 |
| 5 | 0 | 55.5 | 5 | 3.10 |

TABLE 9
Enantioselective hydrogenation of 1 and 3 catalyzed by
N1•Rh in methanol under H₂ (1 atm) at 25° C.
as a function of uniaxial compression.

| substrate | compression (%) | R (%) | S(%) | ee (%) | er |
|---|---|---|---|---|---|
| 1 | 0 | 67.5 | 32.5 | 35 | 2.08 |
| 1 | 30 | 78 | 22 | 56 | 3.55 |
| 1 | 50 | 82.2 | 17.8 | 64.4 | 4.62 |
| 3 | 0 | 65.5 | 34.5 | 31 | 1.90 |
| 3 | 30 | 75 | 25 | 50 | 3.00 |
| 3 | 50 | 78 | 22 | 56 | 3.55 |

### Example 4

### Computational Analysis

All calculations were performed with Gaussian 16.C in vacuum using the Berny algorithm to locate stationary points with tight convergence criteria and ultrafine integration grids. Conformational ensembles of all kinetically significant stationary states were built systematically as previously described except with the MN15/def2TSV model chemistry. MN15 was chosen because it was previously recommended for calculation of reaction barriers in organometallic complexes of transition metals (Yu et al. Chem. Sci. 2016, 7 (8), 5032-5051). The wavefunction stability of the converged geometries of all transition state was confirmed with the Stable test. Geometries under extrinsic force were optimized using the iop(1/165) overlay procedure with very soft spring constant (10⁻⁶ Hartree/Bohr). All stationary states were optimized (both diastereomers of CS through Int4) at 0, 0.5 and 2 nN and product-determining states (both diastereomers of CS, TS1, Int1, TS2) additionally at 0.1, 0.25, 0.75, 1, 1.5 and 2.5 nN at MN15/def2TSV. The latter states were then reoptimized at MN15/def2TZVP.

Thermodynamic corrections were calculated in the ideal-gas/rigid-rotor/pseudo-harmonic oscillator approximation after replacing all frequencies <500 cm⁻¹ with 500 cm⁻¹ to avoid artifactually large contributions of such modes to entropy, as previously recommended (Cramer, C. J. Essentials of Computational Chemistry; Wiley, 2004). Using analytical frequency calculations on converged geometries coupled to an external spring to estimate thermodynamic corrections is theoretically valid because the molecule+its coupled spring comprise a stationary state (Kucharsk et al. J. Mater. Chem. 2011, 21, 8237-8255). Free energies at def2TZVP were calculated by adding electronic energies at def2TZVP to thermodynamic corrections derived for converged geometries at def2TZV because analytical frequency calculations at def2TZVP were unaffordably costly.

Kinetic modelling was performed by numerical integration of the corresponding differential rate laws using the ode85 function of Matlab, assuming that the 2 diastereomeric CS complexes are always in thermodynamic equilibrium and each TS2 is traversed irreversibly, i.e., in the simulations the ratio of the concentrations of Int2, [RS-Int2]/[SS-Int 2], always equaled the ratio of the concentrations of the products, [R-product]/[S-product].

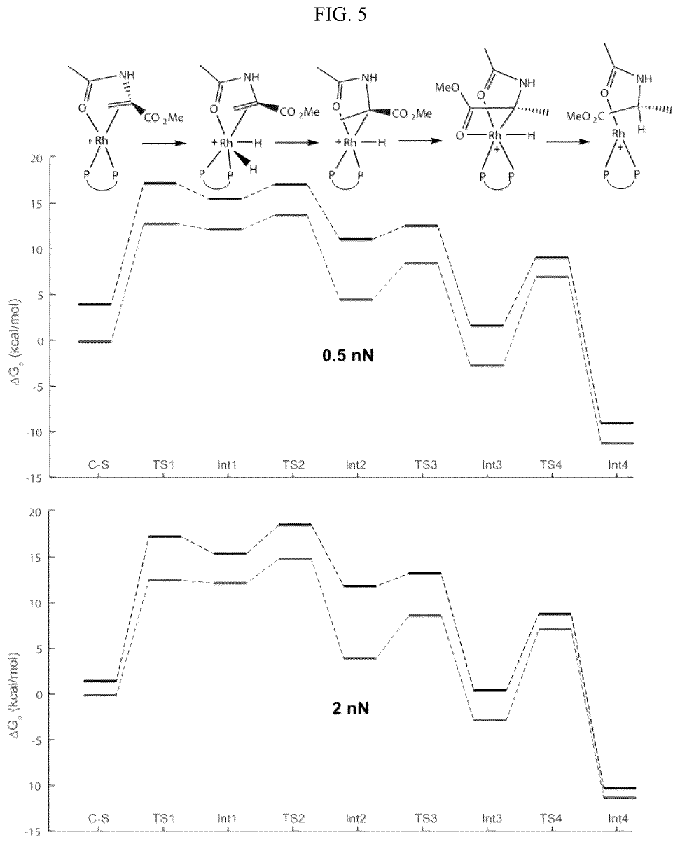

A calculated reaction mechanism is shown in FIG. 5.

### Example 5

### Results and Discussion

Below are further descriptions of the experiments conducted using materials synthesized and characterized as set forth above, and additional results and discussion.

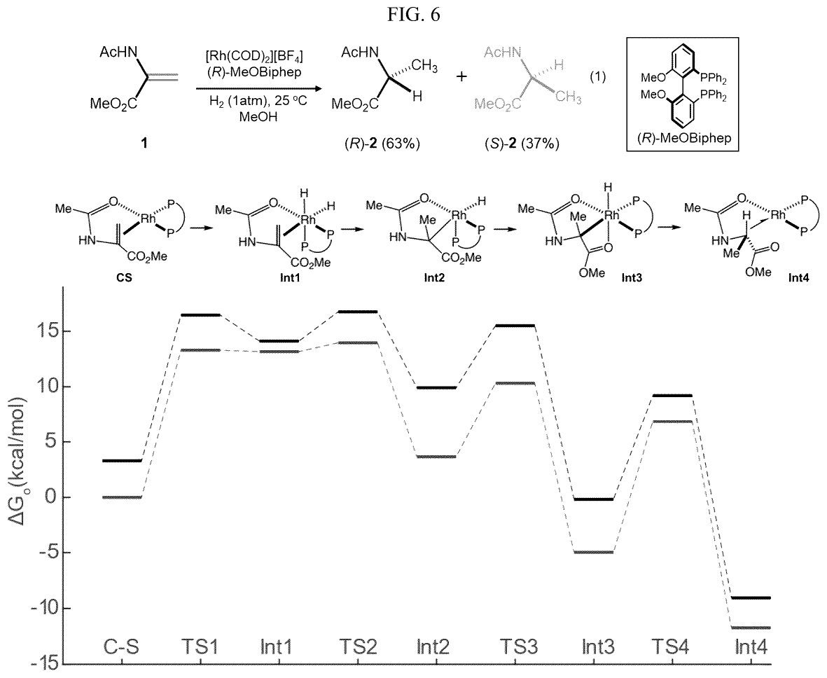

The rhodium (I)-catalyzed enantioselective hydrogenation of methyl 2-amidoacrylate (1) to yield methyl acetylalaninate (2; FIG. 6) is an attractive system to illustrate MMC for two reasons. First, high turnovers, fast rates, and broad solvent compatibility of the reaction facilitate its extension into nontraditional reaction media. Second, the predominate stereoisomer product, as well as the selectivity for that stereoisomer's formation, are influenced heavily by the ligand around Rh(Tang et al. Chem. Rev. 103, 3029-3070 (2003); Xie et al. Chem. Rev. 111, 1713-1760 (2011)). Although high selectivity for the hydrogenation of 1 is realized with a number of chiral phosphine ligands (Halpern, Science 217, 401-407 (1982); Landis et al. J. Am. Chem. Soc. 109, 1746-1754 (1987); Landis et al. J. Am. Chem. Soc. 121, 8741-8754 (1999); Gridnev et al. J. Am. Chem. Soc. 130, 2560-2572 (2008)), a ligand scaffold with modest initial selectivity was used in order to make potential MMC effects more easily detectable. When (R)-MeOBiphep is used as a ligand, a mixture of [Rh(COD)₂][BF₄] (1.25 mM, 1 mol %), bisphosphine ligand (2.50 mM, 2 mol %), and 1 (0.125 M) in MeOH under H₂ (1 atm) at 25° C. for 24 h converts 1 to 2 in >99% yield with an enantiomeric ratio (er) of 63:37 for R and S enantiomers (FIG. 6). Throughout this disclosure, er is used rather than enantiomeric excess (ee) because its direct correlation with the relative energies of the R and S pathways facilitates a comparison of force effects across bulk supports and small molecule systems.

Combined experimental and computational analyses of reaction (1) catalyzed by (MeOBiphep)Rh⁺ (FIG. 6) are consistent with extensive prior studies of the enantioselective hydrogenation of α-acylaminoacrylates catalyzed by rhodium bis(diarylphosphine) complexes (Halpern 1982; Landis 1987; Landis et al. J. Am. Chem. Soc. 121, 8741-8754 (1999); Gridnev et al J. Am. Chem. Soc. 130, 2560-2572 (2008)). This body of work supports a general mechanism involving reversible complexation of substrate to the solvated (P—P)Rh⁺ complex followed by either irreversible oxidative addition of H₂ to the diastereomeric (P—P)Rh(sub)⁺ complexes or reversible (Halpern 1982) oxidative addition followed irreversible migratory insertion of the (P—P)Rh(H)₂(sub)⁺ complexes (Halpern 1982; Landis 1987; Landis 1999; Gridnev 2008). The enantioselectivity of the hydrogenation of 1 catalyzed by (MeOBiphep)Rh⁺ depends negligibly on p(H₂), increasing from 63:37 er to 64:36 er as p (H₂) increased from 0.7 to 4.3 bar, which indicates that pre-equilibrium conditions of the diastereomeric rhodium-enamide complexes are largely realized (Landis 1987). The observed enantioselectivity of hydrogenation therefore reflects the relative kinetics of the conversion of the complex CS (FIG. 6) to Int2, which involves contributions of two elementary steps: the reversible conversion of CS to Int1 and irreversible conversion of Int1 to Int2.

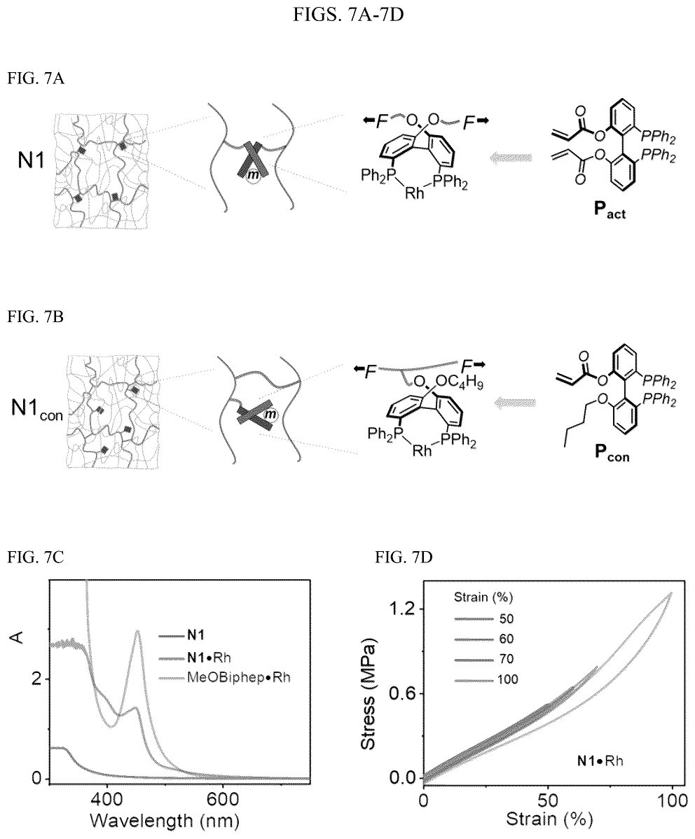

To test whether force-coupled changes in enantioselectivity could be realized to the bulk, supports that can be stretched to high strains without breaking, and in which the ligands are in locations that are prone to high tension, were sought. The strategy employs double network (DN) gels (Ducrot et al. Science 344, 186-189 (2014); Millereau et al. Proc. Natl. Acad. Sci. 115, 9110-9115 (2018)) (FIG. 7A) that comprise a first network that is pre-stretched and brittle due to swelling. An interpenetrating second, soft and elastic network is then formed within this pre-stretched network. When the DN gel is stretched, the force is concentrated in the pre-stretched first network (Matsuda et al. Science 363, 504-508 (2019); Wang et al. Science 374, 193-196 (2021)). The MMC was therefore embedded into the crosslinker of the first network. As the first network is increasingly strained, some strands within it break, but the interpenetrating second network holds the DN together by absorbing stress that is transferred due to strand scission in the first network (Matsuda 2019; Wang 2021). As a result, DNs are typically tougher than either the first or second single network alone, and they could allow greater tension to be applied to the bound catalysts. The first network was made by free radical addition polymerization of ethyl acrylate (50 wt %) and the mechanically active phosphine crosslinker (R)-6,6′-bis(diphenylphosphaneyl)-[1,1′-biphenyl]-2,2′-diyl diacrylate (P_{act}; 0.025 eq, FIG. 7A) in toluene. The second network was introduced by swelling the first network in methyl acrylate with 1,4-butanediol diacrylate crosslinker (0.005 mol %) and polymerizing those components within the first network. Following polymerization, the resulting metal-free DN (N1) was extracted three times by toluene to remove residual monomer or free oligomer.

Subsequently, rhodium (I) was loaded into nine replicate batches of metal-free N1 with [Rh(COD)₂][BF₄] to prepare the polymer-supported N1·Rh for hydrogenation of methyl 2-acetamidoacrylate (1), and these putative MMC supports were characterized by UV-Vis spectroscopy and tensile testing. As shown in FIG. 7C, compared to metal-free N1 (gray curve), the Rh-loaded DN gel N1·Rh displays a distinct absorption band at 450 nm (blue curve), which is similar to [(MeOBiphep)Rh(COD)]⁺BF₄⁻ in solution (orange curve), indicating that intact (P_{act})Rh MMCs are formed within the DN. The N1·Rh DNs can be stretched under cyclic loading up to 200% strain, and there is no obvious hysteresis on the timescale of minutes below strains of approximately 70% (FIG. 7D). The hysteresis at higher strain is attributed to covalent bond scission in the primary network (Ducrot 2014; Webber et al. Macromolecules 40, 2919-2927 (2007)), which in turn suggests that maximum-activity sustained MMC in N1·Rh requires strains less than but close to 70%. At higher strains, the material tear limited the reaction extents.

The enantioselectivity of reaction (1) in the bulk was investigated in nine replicate batches of N1·Rh(containing mechanically active P_{act}) that were stretched manually to different strains in a homemade clamp and immersed in solution. The reaction conditions were similar to those employed in the small molecule system with (R)-MeOBiphep used as a ligand, except that the substrate concentration was increased to compensate for the reduced transport through the network and the overall dilution of the submerged network by added, surrounding solvent. Notably, the initial er of the as-formed network (er₀) varied from one network to the next (72:28 to 76:24), providing an opportunity to test how robust any observed MMC effect is across variations in network swelling and internal environment. The strain of the stretched networks was calculated as ε=(L_{f}−Lᵢ)/Lᵢ, where Lᵢ is the initial distance between the clamps and L_{f} is the final distance.

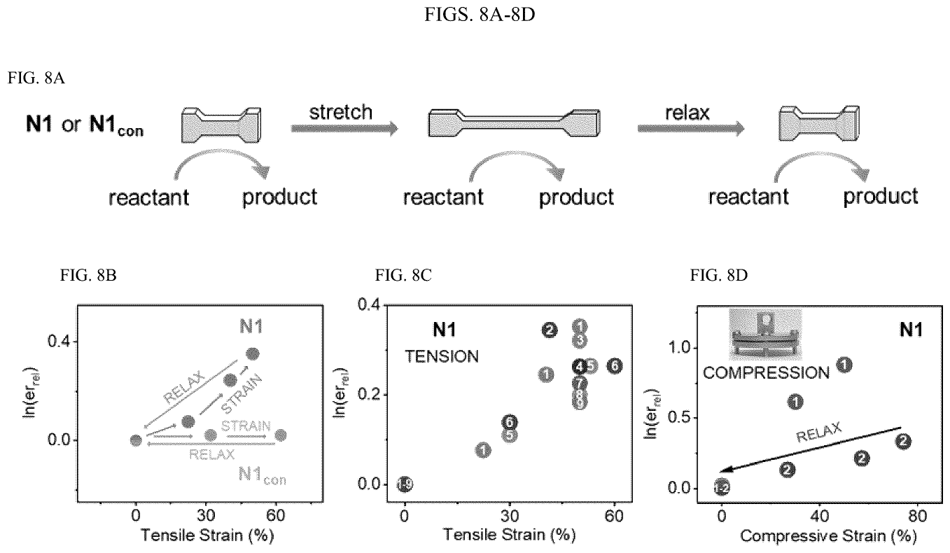

The change in enantioselectivity was quantified by measuring enantiomeric ratio at each applied strain (era), (Table 10, entries 1-5; FIG. 8A). As shown in FIG. 8B, erᵣₑₗ=er_{ε}/er₀ produced by a single MMC support increases with ε, to values of 1.09, 1.29, and 1.42 at ε=22, 40, and 50%, respectively, and it returns to its initial value (erᵣₑₗ=1) when the support is returned to its initial state. Multiple batches of N1·Rh(FIG. 8C), as well as in an MMC with a lower P_{act} content of 1 mol % (N2·Rh) (FIG. 3), manifest the same positive dependence of er on ε. Similar strain-dependent and reversible modulation of enantioselectivity is also observed under uniaxial compression of N1·Rh between parallel plates (FIG. 8D), in one case resulting in the maximum MMC effect observed across all experiments: an erᵣₑₗ of 2.3 when compressed to approximately 50% strain (corresponding to a change in ee of 35% to 65%).

TABLE 10
Enantioselectivity of Rh(I)-Catalyzed Hydrogenation of
Reaction (1) as a Function of Applied Tensile Strain in Loaded
and Unloaded N1•Rh and N1_{con}•Rh (single gel of each type).
Reactions Conducted in MeOH at 25° C. and 1 atm H₂.
Uncertainty in strain is ±2%.

| | | Strain | Yield | |
|---|---|---|---|---|
| Entry | Network | (%) | (%) | er |
| 1 | N1•Rh | 0 | 77 | 75:25 |
| 2 | N1•Rh | 22 | 34 | 76:24 |
| 3 | N1•Rh | 40 | 21 | 79.5:20.5 |
| 4 | N1•Rh | 50 | 44 | 81:19 |
| 5 | N1•Rh | relaxed | 31 | 75:25 |
| | | to 0 | | |
| 6 | N1_{con}•Rh | 0 | 42 | 63.5:36.5 |
| 7 | N1_{con}•Rh | 32 | 38 | 64:36 |
| 8 | N1_{con}•Rh | 62 | 17 | 64:36 |
| 9 | N1_{con}•Rh | relaxed | 13 | 63.5:36.5 |
| | | to 0 | | |

To confirm that the enhanced enantioselectivity is due to coupled tension, the control network N1_{con}·Rh was synthesized, in which a phosphine control ligand (P_{con}) is covalently linked to the polymer through a single site (FIG. 7B). The dihedral angle of the ligand in the resulting catalyst is therefore mechanically decoupled from tension in the polymer support. Under the same reaction conditions and comparable range of applied strain, a negligible change in erᵣₑₗ with strain was found (erᵣₑₗ=1.02 at ε=62%) (Table 10, entries 6-9; FIG. 8B), supporting a mechanical origin to the improved selectivity observed in N1·Rh (Kelber et al. Angew. Chem., Int. Ed. 59, 23283-23290 (2020)).

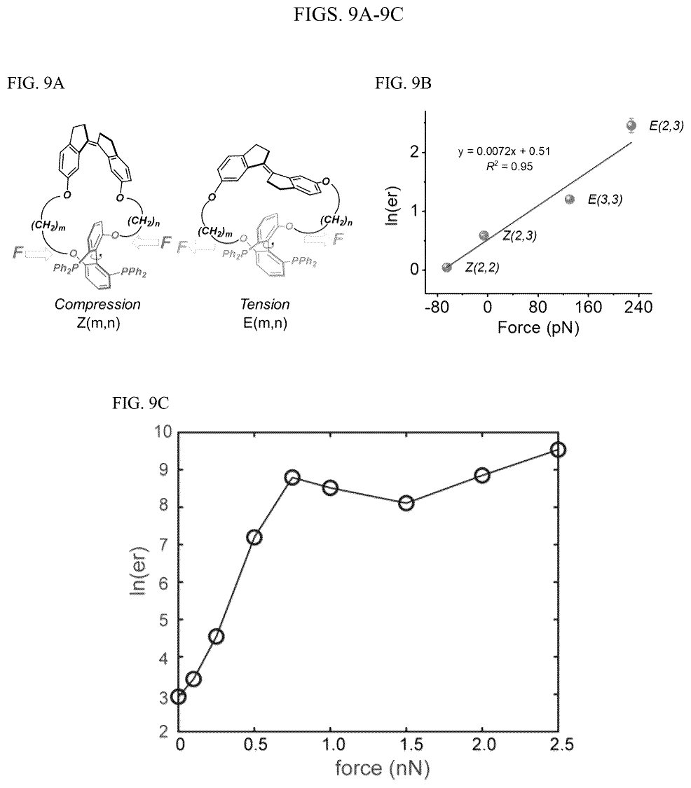

To gain quantitative insights into the effect of force on reaction outcome, a series of strained macrocycles that has been employed previously was used (Kean 2014; Wang 2020; Yu 2021; Yu 2022). Macrocyclic molecular force probes E(m,n) and Z(m,n) comprise stiff stilbene (1,1′-biindane) tethered to the MeOBiphep moiety. These strained macrocycles subject the oxygen atoms of the bisphosphine moiety to a compressive or stretching force, respectively, whose magnitude is controlled by the tether length and the stereoisomer (E vs. Z) of the stiff stilbene (FIG. 9A). Previous work has shown that the effect of force applied intramolecularly and quantified in this way is indistinguishable to that applied in a strained polymer (Akbulatov et al. J. Am. Chem. Soc. 134, 7620-7623 (2012)). The tension is coupled to the dihedral angle of the chiral MeOBiphep ligand, and that coupling has been demonstrated to influence the rate or selectivity of other reaction classes (Kean 2014; Wang 2020; Yu 2021; Yu 2022). The change in product selectivity observed experimentally is determined as a function of force that has been calculated previously to vary from approximately −65 pN for Z(2,2) to 228 pN for the macrocyclic ligands (Table 11, FIG. 9B). The molecular force probe (MFP) ligands therefore provide a convenient method to apply force to metal complexes and to quantify the effect of that force on product selectivity using conventional reaction conditions and spectroscopic methods, and these experimental methods are supported and extended by calculations of the force dependence.

The hydrogenation of 1 catalyzed by rhodium MFP complexes was studied under the same conditions applied to the force-free MeOBiphep system (FIG. 6). As shown in FIG. 9B and Table 11, the enantioselectivity of reaction (1) improves as more force of extension is applied to the Biphep dihedral. The largely unaltered Z(2,3) ligand (−6 pN) yields a R: S ratio of 64:36 (Table 11, entry 2), which is similar to that obtained using MeOBiphep (er of 63:37). In comparison, a compressive force of −65 pN applied via the Z(2,2) ligand lowers the selectively to R:S=51:49 (Table 11, entry 1), whereas forces of tension increase it to 77:23 (Table 11, entry 3) and 92:8 (Table 11, entry 4) at 130 pN [E(3,3)] and 228 pN [E(2,3)], respectively. As shown in Tables S2 and S3, the enantioselectivity does not change with conversion, and it is similarly independent of ligand concentration above 5.0 mM. The difference in apparent activation energies of the competing reaction paths to products (R)-2 and (S)-2 is given by RT·ln(er). When ln(er) is plotted as a function of force (FIG. 9B), the data are consistent with a linear relationship between the difference in the activation energies of the competing pathways and force. A linear relationship between activation barrier and force appears to be a common feature of these complexes, having been observed previously when the same ligands modulate the oxidative addition or reductive elimination at Pd(0) and Pt(II) centers (Wang 2020; Yu 2021), respectively; the semilog correlation observed in FIG. 9B is consistent with this trend. FIG. 9B therefore provides a useful calibration curve for interpreting force-coupled effects in the strained elastomeric supports of interest, and calculated force-coupled enantioselectivity based on the activation energies of the competing hydrogenation steps that align with the experimental dependence allow further extrapolation to high forces (FIG. 9C). The computational force dependence includes contributions from each of the elementary steps that contribute to the net conversion of the rhodium-enamide complex CS to hydride insertion product Int2 (FIG. 6), but the predominant contribution comes from a slowing of the pro-S and acceleration of the pro-R insertion (TS2, FIG. 6) with increasing force.

TABLE 11
Enantioselectivity of Rh(I)-Catalyzed Hydrogenation
of reaction (1) as a Function of Ligand Restoring Force in
MeOH at 25° C. under H₂ (1 atm) for 24 h.

| | | | | | | | est. force |
|---|---|---|---|---|---|---|---|
| entry | MFPs | yield (%) | R (%) | S (%) | er | ee (%) | (pN) |
| 1 | Z(2, 2) | >99 | 51 | 49 | 1.04 | 2 | −65 |
| 2 | Z(2, 3) | >99 | 64 | 36 | 1.78 | 28 | −6 |
| 3 | E(3, 3) | >99 | 77 | 23 | 3.35 | 54 | 130 |
| 4 | E(2, 3) | >99 | 92 | 8 | 11.50 | 84 | 228 |

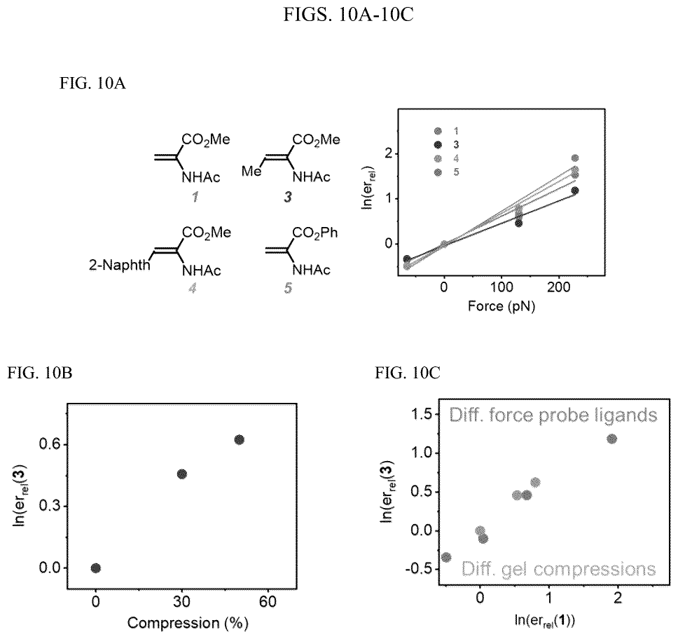

Finally, the force effects were explored to determine whether they translate to additional 2-acetamidoacrylates (3-5). The er for all three substrates increases with force in the same manner as 1, although the force dependence varies (FIG. 10A). The least sensitive substrate 3 was chosen for evaluation in MMC; it also demonstrates improved selectivity upon compression of the bulk N1·Rh(FIG. 10B). Importantly, when erᵣₑₗ for the least sensitive substrate 3 is plotted against erᵣₑₗ for 1 across all conditions examined, the relative selectivity in the force probe ligands and the strained elastomers collapse onto a single free energy relationship that is consistent with a common underlying effector (force) across the various macrocycles and networks (FIG. 10C).

The effects reported here point to the potential of MMC to complement the use of molecular switches and machines (Pizzolato et al. Nat. Catal. 3, 488-496 (2020); Biagini et al. Angew. Chem. 131, 9981-9985 (2019)) in organic synthesis. MMC can be achieved using a material and device that are immediately accessible in almost any chemistry laboratory. The enantioselectivity enhancement achieved by us so far likely reflects the existing limits on the collective capacity to control through molecular design the distribution of macroscopic load across the network rather than an intrinsic limit of MMC. The macrocyclic bisphosphine ligands show that force differences of approximately 300 pN improve er by an order of magnitude (FIG. 9C), and computations predict the same scaling to persist up to 1 nN (FIG. 9C). For calibration, the average change in ln(er) observed for the hydrogenation of 1 in N1·Rh stretched to 50% strain is 0.35±0.18 (FIG. 8B), and the change in ln(er) with added force obtained from the molecular force probe ligands is 7.2×10⁻³ pN⁻¹ (slope of best linear fit to the data in FIG. 9B). The ratio of these two values suggests that the average embedded P_{act} at 50% strain is reacting under force on the order of 50 pN. A small fraction of P_{act}-containing strands within the network likely experiences forces much greater than 50 pN, but the net effect from these much more selective catalysts may be cancelled by counterparts that experience lower forces or are entirely uncoupled from macroscopic tension due to network defects (Zhong et al. Science 353, 1264-1268 (2016)). Thus, improved network designs that either focus stress on the MMC component or distribute force more evenly will increase MMC effects. Additional gains can be realized through device engineering. For example, forces of over the 1 nN threshold necessary for 300-fold improvements are easily delivered to single macromolecules using atomic force microscope techniques (Wang et al. Synlett 33, 885-889 (2022)), and the results reported here motivate the development of other device architectures that can deliver similar forces to larger arrays of single molecules or ensembles of a few strands.

### Example 6

### Copper-Catalyzed Reductive Coupling Reactions

General Methods. All reactions were performed under a nitrogen atmosphere in oven-dried glassware employing standard Schlenk or glovebox techniques unless noted otherwise. Nitrogen-flushed plastic syringes and oven-dried stainless-steel cannulas were employed for reagent transfer. Flash chromatography was performed employing 200-400 mesh silica gel 60 (EM). Thin layer chromatography was performed on silica gel 60 F254. NMR spectra were obtained on a Varian INOVA (400 MHZ), Bruker (500 MHz), or Varian INOVA (500 MHz) spectrometer at 25° C. unless otherwise noted. ¹H and ¹³C chemical shifts are referenced to the solvent residual peaks relative to tetramethylsilane (TMS). ¹⁹F was referenced using absolute frequency referencing in Mnova software. Gas chromatography (GC) was performed on a Shimadzu GCMS-QP2010 equipped with a Supelco β-Dex 225 (30 m×0.25 mm×0.25 μm) column using TIC detection. Chiral high performance liquid chromatography (HPLC) was performed on a Shimadzu Prominence Modular HPLC instrument using 4.6×250 mm columns. Racemic samples of 2b, 2a and 3c for HPLC analysis were obtained using rac-Binap as the ligand.

Anhydrous solvents were obtained either from Sigma-Aldrich in Sure/Seal™ containers and used as received or were dried and degassed using an Innovative Technologies PureSolv solvent purification system. All deuterated solvents were obtained from Cambridge Isotope Laboratory and were dried over activated 3 Å molecular sieves. Freshly opened anhydrous CD₂Cl₂ was dried over CaH₂, degassed via three freeze-pump-thaw cycles and stored in a glovebox. (R)-MeOBiphep and all other reagents were purchased from major chemical suppliers and used as received unless otherwise noted. All macrocyclic bisphosphine ligands E/Z(m,n) possessed an R-configuration about the MeOBiphep axis and were synthesized employing published procedures (Kean 2014; Wang 2020). Double network gels N1 and N1_{con} were synthesized employing the procedures set forth above.

### Catalytic Reactions

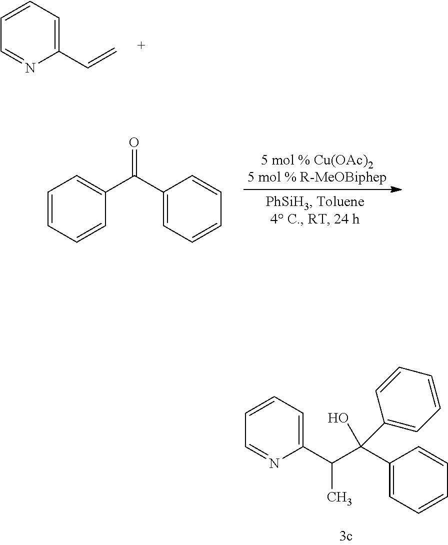

Copper-catalyzed reductive coupling reaction using benzophenone. A solution of 2-vinylpyridine (421 mg, 4.01 mmol), benzophenone (802 mg, 4.40 mmol), Cu(OAc)₂ (36 mg, 0.20 mmol), and R-MeOBiphep; (117 mg, 0.201 mmol) in toluene (20 mL) was stirred at 4° C. for 15 min. PhSiH₃ (590 μL, 4.8 mmol) was added dropwise over 1 min to the solution and the resulting mixture was stirred at 4° C. for 30 min and at room temperature for 24 h. The reaction mixture was treated with silica gel (1.5 g), and the resulting suspension was stirred for 15 min and filtered through a short plug of silica gel and eluted with EtOAc (50 mL). The filtrate was concentrated under vacuum and the resulting residue was chromatographed (SiO₂: 10% EtOAc/hexane) to give 3c (850 mg, 74%) as white solid. The enantiomeric purity of 3c (49.9% ee) was determined by HPLC equipped with a Chiralcel OD-H column (99.5:0.5 hexane: i-PrOH, 0.8 mL/min, 254 nm, 25° C.); tᵣ (major)=10.6 min, tᵣ (minor)=11.4 min. ¹H NMR (500 MHZ, CDCl₃): δ 8.33 (d, J=4.2 Hz, 1H), 7.63 (m, 2H), 7.56-7.49 (td, J=8.8, 1.3 Hz, 2H), 7.47 (dd J=8.5, 1.2 Hz, 2H), 7.32 (t, J=7.6 Hz, 2H), 7.17 (m, 2H), 7.08 (t, J=7.6 Hz, 2H), 7.01 (dd, J=5.9, 5.3 Hz, 2H), 6.96 (m, 1H), 4.02 (q, J=6.2 Hz, 1H), 1.27 (d, J=7.0 Hz, 3H). ¹³C{¹H} NMR (176 MHz, CDCl₃): δ 165.8, 148.6, 148.0, 146.4, 136.9, 128.0, 127.7, 125.9 (d, J=68.3 Hz), 125.8 (d, J=74.6 Hz), 124.1, 121.2, 79.9, 47.3, 17.6. HRMS (ESI) calcd (found) for C₂₀H₁₉NO [MH]⁺: 290.1539 (290.1546).

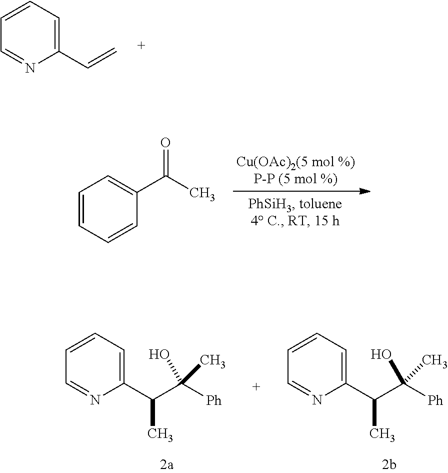

Copper-catalyzed reductive coupling of 2-vinylpyridine (Saxena et al. J. Am. Chem. Soc, 2012, 134 (20), 8428-8431). A solution of 2-vinylpyridine (41 mg, 0.39 mmol), acetophenone (52 mg, 0.43 mmol), Cu(OAc)₂ (3.6 mg, 0.020 mmol), and (R)-MeOBiphep (12 mg, 0.020 mmol) in toluene (2 mL) was stirred at 4° C. for 15 min. PhSiH₃ (59 μL, 0.48 mmol, 1.2 eq) was then added dropwise via syringe over 1 min to the solution. The reaction mixture was stirred at 4° C. for 30 min and at room temperature for 24 h. The reaction mixture was treated with silica gel (200 mg) and the resulting suspension was stirred for 15 min and then filtered through a short plug of silica gel eluting with EtOAc (10 mL). The filtrate was concentrated under vacuum and the resulting residue was chromatographed (SiO₂; 10-15% EtOAc/hexane) to give 2b (7.4 mg, 27% yield) as white solid and 2a (22 mg, 36% yield) as colorless oil. The diastereomeric ratio was determined via ¹H NMR analysis of the crude reaction mixture by integrating the aromatic proton at δ 8.36 for 2a and δ 8.56 for 2b. The enantiomeric purity of a (67.4% ee) was determined by HPLC equipped with a Chiralpak AD-H column (98:2 hexane: i-PrOH 0.8 mL/min, 254 nm, 25° C.); tᵣ (minor)=12.2 min, tᵣ (major)=13.4 min. The enantiomeric purity of 2b (71.9% ee) was determined by HPLC equipped with a Chiralcel OD-H column (98:2 hexane: i-PrOH, 0.8 mL/min, 280 nm, 25° C.); tᵣ (major)=9.8 min, tᵣ (minor)=11.5 min. The ¹H NMR spectra of 2a and 2b were consistent with published spectra. The copper-catalyzed reductive coupling of 2-vinylpyridine with acetophenone employing force probe ligands Z(2,2), Z(3,3), E(2,3), and E(3,3) was performed employing analogous procedures.

### Stereochemical Analyses

Relative configuration of 2a and 2b. The relative configurations of diastereomers 2a and 2b were assigned as R,R/S,S and S,R/R,S, respectively, on the basis of X-ray crystallographic analysis of racemic 2b obtained via vapor diffusion of hexane into an EtOAc solution of rac-2b.

Absolute configuration of 2a. The major enantiomer of a formed in the Cu-catalyzed reductive coupling of 2-vinylpyridine and acetophenone employing R-bisphosphine ligand was assigned as (S,S)-2a on the basis of X-ray crystallographic analysis of the Mosher ester of the minor enantiomer (R,R)-2a.

Triethylamine (167 mg, 1.65 mmol) and DMAP (68 mg, 0.55 mmol) were added via syringe to a solution of(S)-3,3,3-trifluoro-2-methoxy-2-phenylpropanoyl chloride (278 mg, 1.11 mmol) and 2a (125 mg, 0.551 mmol, 67.4% ee, 83.7:16.3 er) in CH₂Cl₂ (0.2 M) at room temperature and the resulting suspension was stirred at room temperature for 48 h. The reaction mixture was washed with water (3×10 mL) and then concentrated under vacuum. The resulting residue was chromatographed (15% EtOAc/hexane) to give (S,S,R)-2a′ (65 mg, 32%, major) as a colorless oil and (R,R,R)-2a″ (11 mg, 28%, minor) as white solid. Mass balance requires that (S,S,R)-2a′ correspond to the major enantiomer of 2a. Vapor diffusion of hexane into a concentrated methylene chloride solution of minor diastereomer (R,R,R)-2a′ gave crystals suitable for X-ray diffraction. X-ray diffraction of (R,R,R)-2a′ established the R,R,R configuration of the minor diastereomer, which established the major enantiomer of 2a formed in the copper-catalyzed coupling of 2-vinylpyridine and acetophenone as (S,S)-2a.

For (S,S,R)-2a′: ¹H NMR (500 MHZ, CDCl₃): δ 8.55 (dt, J=4.7, 0.9 Hz, 1H), 7.48 (td, J=7.7, 1.9 Hz, 1H), 7.41 (m, 1H), 7.35 (m, 4H), 7.31 (m, 3H), 7.19 (m, 2H), 7.13 (ddd, J=7.4, 4.9, 0.8 Hz, 1H), 7.06 (d, J=7.8 Hz, 1H), 3.38 (q, J=7.2 Hz, 1H), 3.22 (d, J=1 Hz, 3H), 2.01 (s, 3H), 1.05 (d, J=7.2 Hz, 3H). ¹⁹{¹H} NMR (471 MHz, CDCl₃): δ−70.6 (s). ¹³C{¹H} NMR (176 MHZ, CDCl₃): δ 162.84, 159.78, 147.45, 141.35, 134.46, 131.52, 128.31, 127.08 (d, J=11.7 Hz), 126.67, 126.16, 124.96 (d, J=4.3 Hz), 124.51, 123.25, 121.60, 120.74, 88.61, 82.92 (q, J=27.2 Hz), 59.37, 54.25, 51.40, 20.01 (d, J=3.8 Hz), 17.08 (d, J=3.6 Hz), 13.37. HRMS (ESI) calcd (found) for C₂₅H₂₄F₃NO₃ [MH]⁺: 444.1781 (444.1781).

For (R,R,R)-2a′: ¹H NMR (500 MHZ, CD₂Cl₂): δ 8.39 (d, J=4.6 Hz, 1H), 7.34 (m, 11H), 7.03 (dd, J=7.3, 5.3 Hz, 1H), 6.99 (d, J=7.9 Hz, 1H), 3.52 (q, J=7.2 Hz, 1H), 3.45 (s, 3H), 1.89 (s, 3H), 1.04 (d, J=7.2 Hz, 3H). ¹⁹{¹H} NMR (471 MHZ, CD₂Cl₂): δ−71.5 (s). ¹³C{¹H} NMR (176 MHZ, CD₂Cl₂): δ 164.25, 160.52, 148.22, 142.55, 135.21, 132.29, 129.29, 128.14, 127.99, 127.51, 127.40, 125.66, 125.19, 124.21, 122.57, 121.56, 89.79, 84.35 (q, J=27.3 Hz), 55.34, 52.09, 29.67, 18.66, 14.36. HRMS (ESI) calcd (found) for C₂₅H₂₄F₃NO₃ [MH]⁺: 444.1781 (444.1778).

Absolute configuration of 2b. The major enantiomer of 2b formed in the Cu-catalyzed reductive coupling of 2-vinylpyridine and acetophenone employing R-bisphosphine ligand was assigned as (S,R)-2b based on X-ray crystallographic analysis of the Mosher ester of (S,R)-2a.

Triethylamine (209 mg, 2.07 mmol) and DMAP (85 mg, 0.69 mmol) were added via syringe to a solution of(S)-3,3,3-trifluoro-2-methoxy-2-phenylpropanoyl chloride (349 mg, 1.38 mmol) and 2b (157 mg, 0.691 mmol, 71.9% ee, 86:14 er) in CH₂Cl₂ (0.2 M) at room temperature and the resulting suspension was stirred at room temperature for 48 h. The reaction mixture was washed with water (3×10 mL) and then concentrated under vacuum. The resulting residue was chromatographed (15% EtOAc/hexane) to give (S,R,R)-2b′ (55 mg, 21%) as a white solid and (R,S,R)-2b′ (4.2 mg, 9.8%) as a colorless oil. Mass balance requires that (S,R,R)-2b′ correspond to the major enantiomer of 2b. Vapor diffusion of hexane into a concentrated ethyl acetate solution of major diastereomer (S,R,R)-2b′ gave crystals suitable for X-ray diffraction. X-ray diffraction of (S,R,R)-2b′ established the S,R,R configuration of the major diastereomer, which established the major enantiomer of 2b formed in the copper-catalyzed coupling of 2-vinylpyridine and acetophenone as (S,R)-2b.

For (S,R,R)-2b′: ¹H NMR (500 MHZ, CDCl₃): δ 8.43 (dt, J=4.8, 0.9 Hz, 1H), 7.57 (m, 2H), 7.41 (dd, J=5.3, 1.8 Hz, 3H), 7.20 (m, 4H), 7.07 (m, 3H), 6.97 (ddd, J=7.4, 4.9, 0.7 Hz, 1H), 6.53 (d, J=7.9 Hz, 1H), 3.62 (d, J=0.2 Hz, 3H), 3.34 (q, J=7.0 Hz, 1H), 1.98 (s, 3H), 1.36 (d, J=7.0 Hz, 3H). ¹⁹{¹H} NMR (471 MHz, CDCl₃): δ−70.7 (s). ¹³C{¹H} NMR (126 MHZ, CDCl₃): δ 164.34, 160.42, 148.38, 142.75, 135.40, 132.20, 129.46, 128.30, 127.89, 127.71, 127.27, 125.10, 124.64, 124.31, 122.35, 121.54, 89.71, 84.54 (q, J=27.6 Hz), 55.46, 53.79, 19.87, 14.63. HRMS (ESI) calcd (found) for C₂₅H₂₄F₃NO₃ [MH]⁺: 444.1781 (444.1773).

For (R,S,R)-2b′: ¹H NMR (500 MHZ, CD₂Cl₂): δ 8.44 (d, J=4.6 Hz, 1H), 7.55 (d, J=7.2 Hz, 2H), 7.51-7.41 (m, 3H), 7.23 (td, J=7.6, 1.7 Hz, 1H), 7.19 (m, 3H), 6.99 (m, 3H), 6.47 (d, J=7.8 Hz, 1H), 3.56 (s, 3H), 3.31 (q, J=7.0 Hz, 1H), 2.08 (s, 3H), 1.41 (d, J=7.0 Hz, 3H). ¹⁹{¹H} NMR (471 MHz, CD₂Cl₂): δ−70.8 (s). ¹³C{¹H} NMR (176 MHZ, CD₂Cl₂): δ 164.27, 160.25, 148.43, 143.00, 135.29, 132.55, 129.58, 128.33, 127.73, 127.39, 127.20, 125.14, 124.55, 124.44, 122.91, 121.48, 89.73, 84.25 (q, J=27.3 Hz), 55.39, 29.67, 19.37, 14.55, 13.97. HRMS (ESI) calcd (found) for C₂₅H₂₄F₃NO₃ [MH]⁺: 444.1781 (444.1778).

Copper-catalyzed asymmetric hydrosilylation of acetophenone (Lipshutz et al. J. Am. Chem. Soc, 2003, 125 (29), 8779-8789). A mixture of CuCl (4.5 mg, 0.045 mmol), ^{t}BuONa (4.3 mg, 0.045 mmol), and bisphosphine ligand [(R)-MeOBiphep, Z(2,2), Z(2,3), E(2,3), E(3,3)] (0.1 mol %) was stirred in toluene (0.5 M) at room temperature for 40 min. Acetophenone (180 mg, 1.5 mmol) was added to the resulting colorless suspension via syringe at 4° C. and the resulting suspension was stirred for 30 min at 4° C. PMHS (430 mg, 7.2 mmol) was added to the reaction mixture via syringe at 4° C., the reaction mixture was stirred for 6 h, and then quenched with aqueous NaOH (10 mL). The biphasic mixture was extracted with diethyl ether (5×20 mL) and the organic extracts were concentrated under vacuum and dried with anhydrous Na₂SO₄. The concentrations of acetophenone, (R)-1-phenylethanol and(S)-1-phenylethanol were determined by GC (Supelco β-Dex 225 30 m×0.25 mm×0.25 μm column using TIC detection) by integrating the peaks at 13.9 min, 15.6 min and 15.9 min, respectively. The GC response factors of acetophenone and rac-1-phenylethanol were determined by GC analysis of equimolar mixtures of authentic samples.

### Catalysis on Solid Supports

Synthesis of N1·Cu, and N1_{con}·Cu. The double network gels N1 and N1_{con} were synthesized as described above. Excised samples of double networks N1 (˜51 mg, ˜2.3 μmol bisphosphine), and N1_{con} (˜50 mg, ˜3.0 μmol bisphosphine) were immersed in solutions of Cu(OAc)₂ (2.0 eq relative to bisphosphine) in methanol (1-2 mL) for ≥3 days at room temperature. The resulting Cu(II)-loaded DNs N1·Cu, and N1_{con}·Cu were immersed in MeOH (2 mL) for ≥2 days to remove unbound Cu with the baths changed every 12 h, and then were immersed in toluene (2 mL) for ≥3 days to remove MeOH with the baths changed every 12 h.

Reductive coupling catalyzed by N1·Cu and N1_{con}·Cu. Catalytic reductive coupling transformations employing Cu-loaded DNs as a function of uniaxial compression were performed employing a previously reported compressing device.^{S5} Cu(II)-loaded DN N1·Cu of dimensions ˜5 mm×˜5 mm×˜1.1 mm (˜63 mg, 4.2 μmol Cu) was secured between the plates of the compressing device and the secured gel and device were then fully immersed in a solution of 2-vinylpyridine (42 mg, 0.40 mmol) and acetophenone (53 mg, 0.44 mmol) in toluene (2 mL) and the solution was stirred at 4° C. for 15 min. PhSiH₃ (59 μL, 0.48 mmol) was then added dropwise to the solution over 1 min. The solution was stirred at 4° C. for 30 min and then at room temperature for 96 h. Silica gel was added to the reaction mixture and the resulting suspension was stirred for 15 min and filtered through a short plug of silica gel eluting with EtOAc (5 mL). The filtrate was concentrated under vacuum and the resulting residue was chromatographed (10% EtOAc/hexane) to give 2a (19 mg, 32%) as colorless oil and 2b (7.5 mg, 24%) as white solid (Table S3). The diastereomeric ratio and enantiomeric ratios of 2a and 2b were determined as described above employing molecular catalysts. Employing analogous procedures, the reductive coupling of 2-vinylpyridine and acetophenone catalyzed N1·Cu was performed at 30%, 50% and 0% compression employing the same sample of N1·Cu, which was rinsed three times with toluene between experiments (Table S3). Reductive coupling of 2-vinylpyridine, acetophenone, and phenylsilane in toluene catalyzed by N1_{con}·Cu as a function of uniaxial compression was performed employing a procedure similar to that employed for the reductive coupling catalyzed by N1·Cu (Table S3) and analyzed by HPLC with the same condition mentioned above.

### Results and Discussion

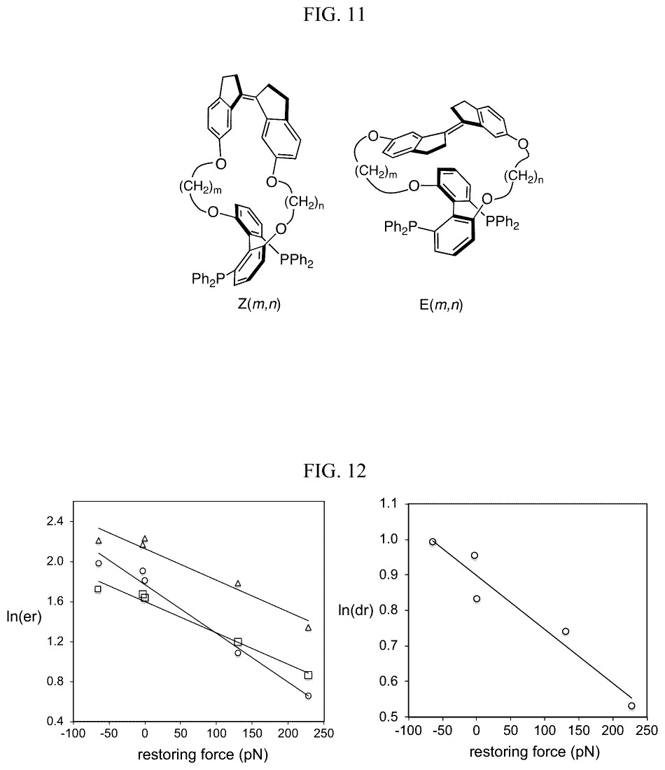

Force modulation of catalytic transformations. The approach to evaluate the effect of force on copper-catalyzed reductions employs the macrocyclic force probe ligands E(m,n) and Z(m,n) (FIG. 11) comprising a 1,1′-biindane photoswitch tethered to the 6,6′ positions of an (R)-Biphep moiety. The Z and E isomers of the 1,1′-biindane apply a compressive or stretching force, respectively, to the oxygen atoms of the bisphosphine moiety. The magnitude of the restoring force is modulated by the tether length and has been quantified by DFT, with untethered (R)-MeOBiphep serving as a force-free benchmark. Within the family of ligands employed in these studies, forces ranging from −65 pN (compressive) to +228 pN (extension) are realized. The applied force similarly modulates the (P)C—C—C—C(P) dihedral angle from ϕ=81° to ϕ=97° across the range of applied forces without perturbation of the electron-donor abilities of the ligands. Previous work has shown that the effect of force applied intramolecularly and quantified in this way is indistinguishable to that applied in a strained polymer.

Copper-catalyzed hydrosilylation. The copper-catalyzed hydrosilylation of ketones displays many of the favorable traits required for multi-state mechanocatalysts on solid supports, most notably the extremely high TOF and TON (10⁻⁶ mol %) and the demonstrated effectiveness of MeOBiphep type ligands. For these reasons, the effect of ligand restoring force on the enantioselectivity of the hydrosilylation of acetophenone catalyzed by copper(I) complexes containing molecular force probe ligands was investigated. In an initial experiment, reaction of acetophenone (0.5 M) with polydimethylsiloxane (5 equiv) catalyzed by a mixture of CuCl (3 mol %), tert-BuONa (3 mol %), and MeOBiphep (0.1 mol %), in toluene at 4° C. for xx h led to formation of 1-phenylethan-1-ol in 99% yield (GC) after NaOH workup (Table 1, entry 1). The enantioselectivity of hydrosilylation was sensitive to ligand restoring force (Table 1). Whereas compressive forces up to −65 pN had no significant effect on the enantioselectivity of hydrosilylation, extension forces of 130 and 228 pN decreased the enantioselectivity of hydrosilylation to 71 and 58% ee, respectively (Table 1).

TABLE 12
Effect of restoring force on the enantioselectivity of copper-catalyzed
hydrosilylation of acetophenone employing force probe ligands.

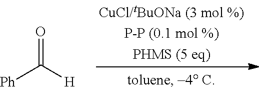

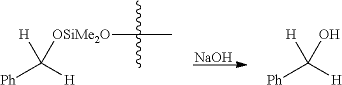

| entry | P-P | Restoring force (pN) | yield (%) | ee (%) |
|---|---|---|---|---|
| 1 | Z (2,2) | −65 | 100 | 80.2 |
| 2 | Z (2,3) | −6 | 99 | 79.4 |
| 3 | MeOBiphep | 0 | 99 | 80.5 |
| 4 | E(3,3) | 130 | 98 | 71.1 |
| 5 | E(2,3) | 228 | 51 | 58.5 |

Copper-catalyzed reductive coupling of 2-vinylpyridine (Saxena 2012). In an initial experiment, reaction of 2-vinylpyridine, acetophenone, and PhSiH₃ catalyzed by a mixture of Cu(OAc)₂ and (R)-MeOBiphep in toluene from 4-25° C. for 24 h led to >99% conversion to form a 2.3:1 mixture of 2a and 2b as determined by ¹H NMR analysis of the crude reaction mixture. Subsequent chromatography led to the isolation of 2a (36%, 67.4% ee) and 2b (27%, 71.9% ee). Over a ˜300 pN range of force from −65 (compression) to +228 (extension) pN, the diastereoselectivity of the reductive coupling of 2-vinylpyridine and acetophenone decreased from 2.7:1 to 1.7:1. Over the same range of extension forces, the enantioselectivity of 2a decreased from 69.7% to 40.7% ee and the enantioselectivity of 2b decreased from 75.7% to 31.8% ee (Table 13). Stereochemical analysis of diastereomers 2a and 2b assigned 2a as 2S,3S/2R,3R diastereomer enriched in the (2S,2S)-2a enantiomer and 2b as the 2R,3S/2S,3R diastereomer enriched in the (2R,3S)-2b enantiomer.

TABLE 13
Reductive coupling of 2-vinylpyridine with acetophenone catalyzed
by a mixture of Cu(OAc)₂ and bisphosphine ligand.

| P-P | force (pN) | convn (%) | 2a:2b | 2a ee (%) | 2b ee (%) |
|---|---|---|---|---|---|
| Z(2,2) | −65 | 95 | 2.7:1 | 69.7 | 75.7 |
| Z(3,3) | −3 | >99 | 2.6:1 | 68.3 | 74.1 |
| (R)-MeOBiphep | 0 | >99 | 2.3:1 | 67.4 | 71.9 |
| E(3,3) | 130 | 95 | 2.1:1 | 53.4 | 49.4 |
| E(2,3) | 228 | >99 | 1.7:1 | 40.7 | 31.8 |

Broadly speaking, the force-dependent rate behavior of transformations occurring within the first coordination sphere of mechanically coupled transition metal complexes can arise from two distinct effects, which are classified as either static or dynamic. Static effects correspond to ground state perturbations of the metal-ligand bond angles and bond lengths, akin to classic ligand bite angle effects, which alter activity by perturbing the relative energies and shapes of the participating molecular orbitals and/or by changing the sterics within the first coordination sphere. Dynamic effects result from mechanochemical coupling of force to the nuclear motions as the complex progresses from ground state to transition state and are proportional to the change in length along the pulling axis between the ground state and the rate-determining transition state.

Previous analysis of force effects on the rates and selectivities of transition metal-mediated and catalyzed transformations including the oxidative addition of bromobenzene to low-ligated palladium(0) complexes, the reductive elimination of biphenyls from nickel and platinum diaryl complexes, the rhodium-catalyzed hydroformylation of 1-alkenes, and the rhodium-catalyzed enantioselective hydrogenation of dehydroamino acids in each case were in accord with the observed force/rate dependence resulting from dynamic effects with no detectable perturbation of ground state geometry. In these cases, the contribution of force to the activation free energy is approximated by −f·Δx, where f is force and Δx is the difference in the molecular distance along the pulling axis (O . . . O in the present case) in the transition state relative to the ground state.

Although there is currently no direct measure of force-dependent perturbation of ground state geometry of copper bisphosphine complexes that catalyzed the hydrosilylation and reductive coupling of acetophenone, the current work is consistent with the predominance of dynamic effects across the range of applied forces. This conclusion is consistent with the roughly linear dependence of the natural logarithm of the observed enantiomeric and diastereomeric ratios on the calculated restoring force (FIG. 12). Of these relationships, the enantiomeric ratio of diastereomer 2b formed in the reductive coupling of 2-vinyl pyridine with acetophenone is more sensitive to force (slope=−4.9×10⁻²) than are the enantiomeric ratio of diastereomer 2a formed in the same reaction (slope=−3.1×10⁻²), the enantiomeric ratio of the hydrosilylation of acetophenone (slope=−3.1×10⁻²), or the diastereomeric ratio of the reductive coupling of 2-vinyl pyridine with acetophenone (slope=−1.5×10⁻²).

Reductive coupling on solid supports. Testing was conducted to determine whether force-coupled changes in the selectivity of copper-catalyzed transformations could be realized in the bulk employing catalysts embedded in elastomeric supports. Macroscopic-to-molecular force transduction requires supports that can be stretched to high strains without breaking, and in which the ligands are in locations that are prone to high tension. To this end, double network (DN) polyacrylate gels have been previously employed to realize macroscopic-to-molecular force transduction for the rhodium-catalyzed hydrogenation of dehydroamino acids. The first network was synthesized via free radical addition polymerization of ethyl acrylate and the mechanically active phosphine crosslinker (R)-6,6′-bis(diphenylphosphaneyl)-[1,1′-biphenyl]-2,2′-diyl diacrylate (P_{act}). The second network was introduced by swelling the first network in methyl acrylate with 1,4-butanediol diacrylate crosslinker and polymerizing those components within the first network. The resulting metal-free DN (N1) was extracted to remove residual monomer and subsequently loaded with rhodium. Because the first network that is pre-stretched and brittle due to swelling while the interpenetrating second network is soft and elastic, when the DN gel is stretched, the force is concentrated in the pre-stretched first network bearing the catalytically active cross linkers.

Employing a similar approach, the mechanically-coupled double network catalyst gel N1·Cu and also the control network N1_{con}·Cu, in which a phosphine control ligand (P_{con}) is covalently linked to the polymer through a single site and therefore mechanically decoupled, were synthesized. The catalytically active DN gel N1·Cu was manually compressed in a bespoke device and fully immersed in a toluene solution of 2-vinylpyridine, acetophenone and PhSiH₃ and the solution was stirred at room temperature for 96 h. The effect of macroscopic strain on the selectivity of the reductive coupling mirrored the observations employing molecular catalysts. The diastereoselectivity of reductive coupling decreased from 1.9 to 1.7 as strain increased from 0 to 50% and then returned to its nascent value when the gel was relaxed (Table 14, entries 1-4). In a similar manner, the enantiopurity of 2a decreased from 48 to 44% and the enantiopurity of 2b decreased from 47 to 42% as the strain was increased from 0- to 50%; both values returned to their nascent value when the gel was relaxed. In comparison, neither the diastereoselectivity nor enantioselectivity of the reductive coupling of acetophenone with 2-vinyl pyridine catalyzed by the mechanically decoupled DN network N1_{con}·Cu was significantly perturbed by 60% strain (Table 14, entries 5 and 6). These observations support the contention that the observed modulation of selectivity with applied strain in the reductive coupling of 2-vinyl pyridine with acetophenone catalyzed by N1·Cu is due to coupled tension.

TABLE 14
Effect of compression on the reductive coupling of 2-vinylpyridine
(0.20M) with acetophenone (0.22M) and phenylsilane (0.24M) catalyzed
by N1•Cu and N1_{con}•Cu in toluene at 25° C.

| | | excess | convn. | 2a ee | 2b ee | |
|---|---|---|---|---|---|---|
| entry | support | strain (%) | (%) | (%) | (%) | 2a:2b |
| 1 | N1•Cu | 0 | >99 | 48.4 | 46.9 | 1.9:1 |
| 2 | N1•Cu | 30 | 90 | 45.8 | 45.0 | 1.8:1 |
| 3 | N1•Cu | 50 | 60 | 44.2 | 42.5 | 1.7:1 |
| 4 | N1•Cu | 0 | >99 | 48.3 | 46.3 | 1.9:1 |
| 5 | N1_{con}•Cu | 0 | >99 | 52.4 | 61.9 | 1.8:1 |
| 6 | N1_{con}•Cu | 60 | 95 | 52.2 | 62.1 | 1.8:1 |

The observed selectivities for the reductive coupling of 2-vinyl pyridine with acetophenone catalyzed by N1·Cu at 0% strain were markedly lower than were the selectivities obtained with the zero-force molecular copper catalyst generated from Cu(OAc)₂ and MeO-Biphep. This was initially attributed to the diminished selectivity in N1·Cu at 0% strain relative to the molecular control to latent strain in the uncompressed DN N1·Cu caused by solvent swelling. Supporting this contention, the selectivities for the reductive coupling of 2-vinyl pyridine with acetophenone catalyzed by the mechanically decoupled DN N1_{con}·Cu were significantly greater than for N1·Cu. However, the selectivities observed for reductive coupling in N1_{con}·Cu are also diminished relative to the molecular zero strain catalysts, which pointed to additional contributions to the diminished selectivities in the DNs. To this point, the potential contribution of (1) medium effects in the swollen DN was considered, owing to the presumably more polar environment relative to the force-free molecular catalyst system and (2) the possible contribution of unligated Cu(OAc)₂. These possibilities were tested by an additional pair of control experiments employing the force-free molecular catalysis system. In one experiment, reductive coupling of 2-vinylpyridene with acetophenone catalyzed by Cu(OAc)₂/MeO-Biphep in a solvent system comprising polymethylacrylate (PMA) in toluene (designed to mimic the medium effects experienced in the solvent-swollen DNs) led to 80% conversion to form a mixture of 2a and 2b with diastereoselectivity and enantioselectivity diminished relative to the reductive coupling catalyzed by Cu(OAc)₂/MeO-Biphep in toluene (Table 15). In a second experiment, reaction of 2-vinylpyridene with acetophenone catalyzed by Cu(OAc)₂ in PMA/toluene with no phosphine ligand led to no consumption of starting materials. Taken together, these experiments argue against the participation of unligated Cu(OAc)₂ in the reductive coupling of 2-vinylpyridene with acetophenone in the DNs and point to solvent medium effects as a contributor to the diminished selectivity of reductive coupling in the DNs relative to the force-free molecular system.

TABLE 15
Reductive coupling of 2-vinylpyridine with acetophenone catalyzed by a mixture of
Cu(OAc)₂ and (R)—MeO-Biphep in toluene containing polymethylacrylate (PMA).

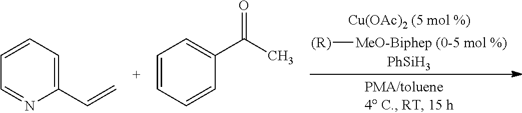

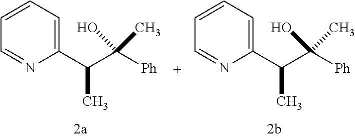

| (R)—MeO-Biphep | convn (%) | 2a:2b | 2a ee (%) | 2b ee (%) |
|---|---|---|---|---|
| 5 mol% | 80 | 1.4:1 | 56.1 | 47.5 |
| — | 0 | — | — | — |

1. A method for conducting a catalytic reaction, comprising:
providing a catalyst covalently bound to a solid support;
conducting the catalytic reaction on the solid support; and
applying a force to the solid support to modify the selectivity of the catalytic reaction.

2. The method of claim 1, wherein the catalyst comprises a metal selected from rhodium, copper, palladium, nickel, platinum, ruthenium, iridium, and cobalt.

3. The method of claim 2, wherein the metal is rhodium or copper.

4. The method of claim 1, wherein the catalyst comprises a ligand that is covalently bound to the solid support via at least two points of attachment.

5. The method of claim 4, wherein the catalyst comprises a bidentate phosphine ligand having two reactive moieties that attach to the solid support.

6. The method of claim 4, wherein the ligand is a compound of formula (I): 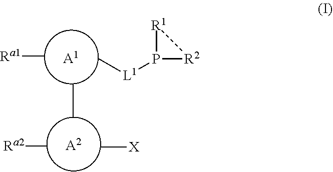
wherein:
A¹ and A² are each independently aryl or heteroaryl;
R^{a1} and R^{a2} are each independently selected from -Q-L-Y, wherein:
Q is —OC(O)—, —NHC(O)—, or O;
L is a bond or —(CH₂)ₙ—, wherein n is 1, 2, 3, 4, 5, or 6; and
Y is a reactive group; 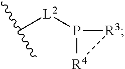
X is hydrogen or a group
L¹ and L² are each independently a bond or —CH₂—; and
R¹, R², R³, and R⁴ are each independently selected from C₁-C₆ alkyl, aryl, heteroaryl, and C₃-C₆ cycloalkyl, each of which is independently unsubstituted or substituted with 1 or 2 substituents, and wherein each dashed line represents the presence or absence of a covalent bond.

7. The method of claim 4, wherein the ligand is selected from:

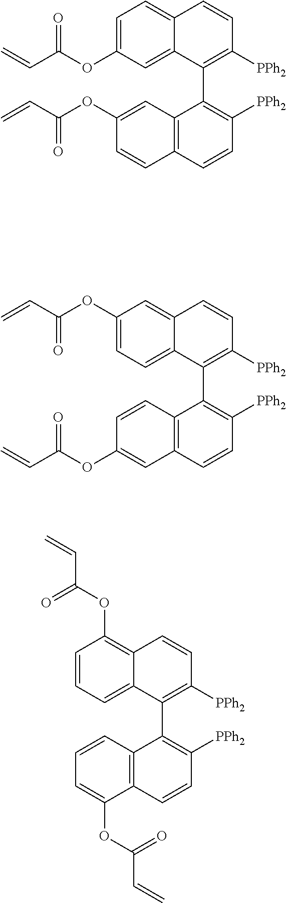

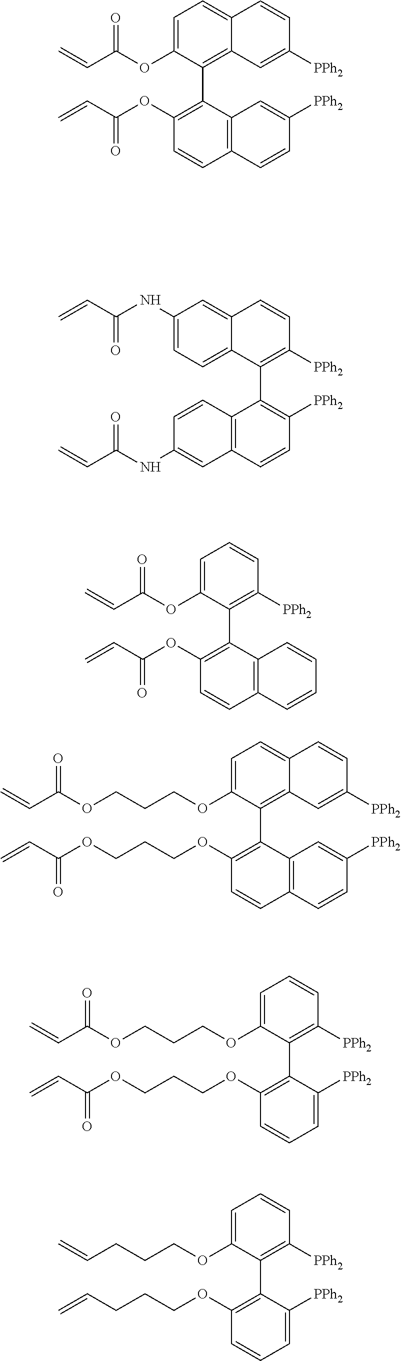

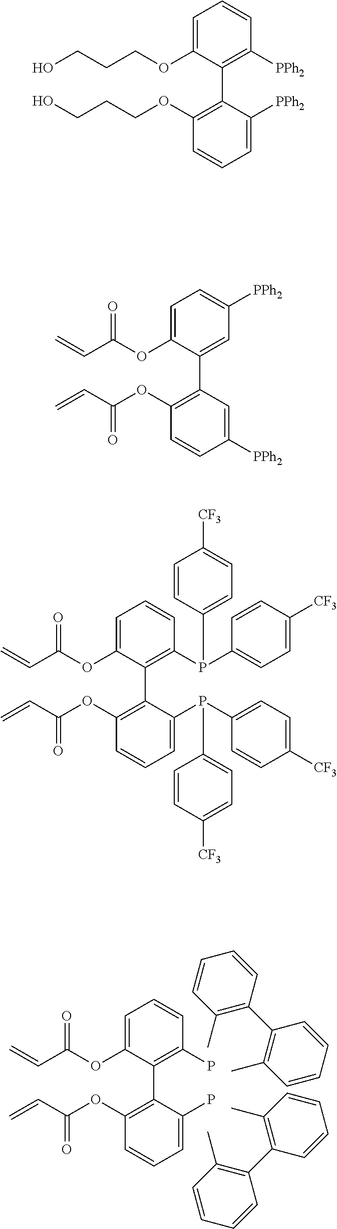

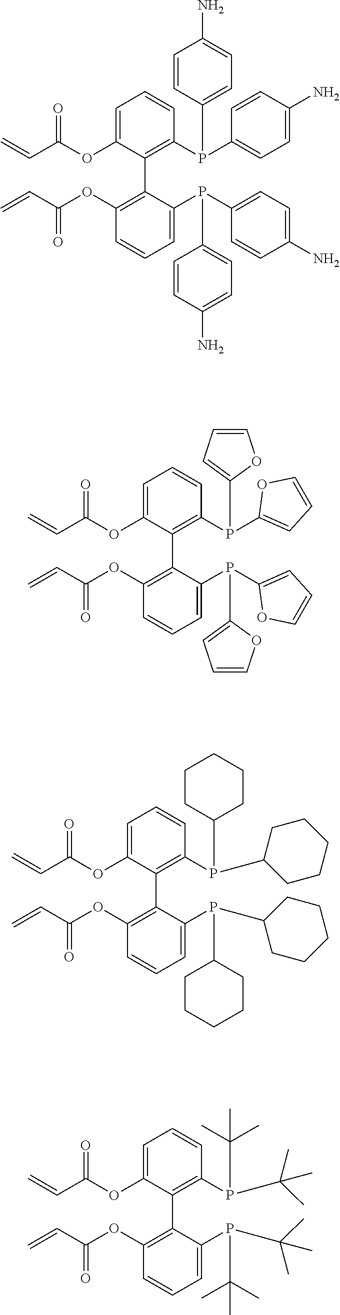 [SMILES: C=CC(=O)Oc1cccc(P(c2ccc(N)cc2)c2ccc(N)cc2)c1-c1c(OC(=O)C=C)cccc1P(c1ccc(N)cc1)c1ccc(N)cc1 ; C=CC(=O)Oc1cccc(P(c2ccco2)c2ccco2)c1-c1c(OC(=O)C=C)cccc1P(c1ccco1)c1ccco1 ; C=CC(=O)Oc1cccc(P(C2CCCCC2)C2CCCCC2)c1-c1c(OC(=O)C=C)cccc1P(C1CCCCC1)C1CCCCC1 ; C=CC(=O)Oc1cccc(P(C(C)(C)C)C(C)(C)C)c1-c1c(OC(=O)C=C)cccc1P(C(C)(C)C)C(C)(C)C]

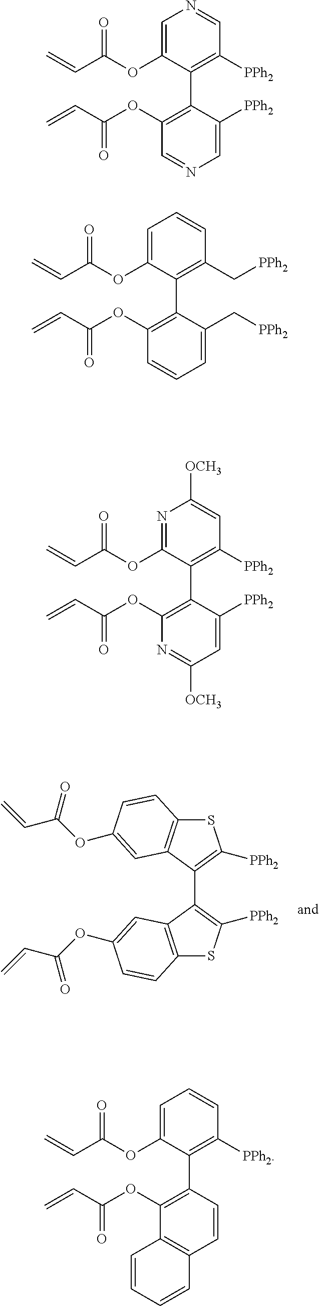

8. The method of claim 1, wherein the solid support is selected from single and double network organogels and hydrogels.

9. The method of claim 1, wherein the solid support comprises a polymer network selected from polysiloxanes, polyurethanes, poly(meth)acrylates, and polyacrylamides.

10. The method of claim 9, wherein the solid support comprises a polyacrylate polymer network.

11. The method of claim 10, wherein the polyacrylate polymer network comprises a poly(alkyl acrylate).

12. The method of claim 11, wherein the poly(alkyl)acrylate is selected from poly(methyl acrylate) and poly(ethyl acrylate).

13. The method of claim 1, wherein the catalytic reaction is selected from a hydrogenation reaction, a carbon-carbon coupling reaction, a hydroformylation reaction, a hydrosilylation reaction, an ethylene alkoxycarbonylation reaction, a hydroboration reaction, a carbonyl allylation reaction, a cycloaddition reaction, and a cyclotrimerization reaction.

14. The method of claim 15, wherein the catalytic reaction is a hydrogenation reaction.

15. The method of claim 1, wherein the step of applying a force to the solid support comprises stretching the solid support.

16. The method of claim 1, wherein the step of applying a force to the solid support comprises compressing the solid support.

17. The method of claim 1, wherein the step of applying a force to the solid support comprises swelling the solid support.

18. The method of claim 17, wherein swelling the solid support comprises contacting the solid support with a second solvent, wherein the second solvent is different from a first solvent used to carry out the catalytic reaction.

19. The method of claim 1, wherein the modification of the selectivity of the catalytic reaction comprises a modification of enantioselectivity, diastereoselectivity, and regioselectivity.

20. A system for conducting a catalytic reaction, comprising:
(a) a solid support;
(b) a catalyst covalently bound to the solid support via at least two points of attachment;
wherein application of an external force to the solid support changes the selectivity of the catalytic reaction.
